(19) United States
(12) Patent Application Publication
(10) Pub. No.: US 2026/0200835 A1
(43) Pub. Date: Jul. 16, 2026

(54) ACETONITRILE COMPOSITIONS AND METHODS OF MAKING THE SAME

(71) Applicants: Nitto Denko Corporation, Osaka (JP); Safion Renewables Inc., Louisville, CO (US)

(72) Inventors: Eric M. KARP, Arvada, CO (US); Jacob Scott KRUGER, Arvada, CO (US); Ryan PRESTANGEN, Denver, CO (US); Lauren Ann Riley DRUMM, Lafayette, CO (US); Andrew John BORCHERT, Arvada, CO (US); Alexander Hunt JENKINS, Boulder, CO (US)

(21) Appl. No.: 19/328,004

(22) Filed: Sep. 12, 2025

Related U.S. Application Data
(60) Provisional application No. 63/708,557, filed on Oct. 17, 2024.

(51) Int. Cl. C07C 253/22; C07C 253/34

(52) U.S. Cl. CPC C07C 253/22; C07C 253/34

(57) ABSTRACT

Provided herein are methods of making acetonitrile from (e.g., biologically produced) precursors. The methods herein may be less carbon intensive and produce fewer toxic byproducts than traditional methods. Also provided herein are acetonitrile compositions, such as prepared by the methods provided herein.

### BACKGROUND

Acetonitrile is an organic solvent used in analytical chemistry and industrial and pharmaceutical manufacturing processes. Historically, acetonitrile has been made on the industrial scale as a byproduct of acrylonitrile production (via the Standard Oil of Ohio (SOHIO) process). The global acetonitrile market stood at approximately 180,000 tonnes in 2022, and is anticipated to continue growing.

### SUMMARY

The SOHIO process relies on the use of fossil fuel-based feedstocks and results in the release of toxic side products. Alternative processes to produce high purity acetonitrile that are more environmentally friendly and less toxic are needed.

Provided herein, in some embodiments, are methods of making acetonitrile (and compositions comprising acetonitrile), also referred to herein as “bio-based acetonitrile”. The methods provided herein are environmentally friendly and less toxic alternatives to the standard propylene ammoxidation (SOHIO) process, which allow for preparation of acetonitrile at high levels of purity (e.g., oligonucleotide synthesis quality or pharmaceutical grade quality). In some embodiments, the methods herein provide for the use of 100% bio-based feedstocks, allowing for a more ecologically sustainable method for preparation of acetonitrile. Additionally, the methods herein are decoupled from production of other reagents (e.g., acrylonitrile) and provide for direct preparation of acetonitrile without generation of explosive hazards in the process or release of toxic byproducts, such as hydrogen cyanide.

Provided herein, in some embodiments, is a method of making an acetonitrile composition, the method comprising:
(a) providing a biologically produced acetonitrile precursor;
(b) purifying the biologically produced acetonitrile precursor;
(c) reacting the biologically produced acetonitrile precursor with a nitrogen source and a catalyst to provide crude acetonitrile; and
(d) purifying the crude acetonitrile to provide the acetonitrile composition, wherein the reacting produces no more than 20 wt % of organic products other than acetonitrile, and wherein the acetonitrile composition comprises less than 2 wt % of impurities.

Provided herein, in some embodiments, is a method of making an acetonitrile composition, the method comprising:
(a) providing a biologically produced acetonitrile precursor;
(b) purifying the biologically produced acetonitrile precursor;
(c) reacting the biologically produced acetonitrile precursor with a nitrogen source and a catalyst to provide crude acetonitrile; and
(d) purifying the crude acetonitrile to provide the acetonitrile composition, wherein the method does not generate hydrogen cyanide.

In some embodiments, the reacting produces no more than 10 wt % of organic products other than acetonitrile.

In some embodiments, the acetonitrile composition comprises less than 2 wt % of impurities. In some embodiments, the acetonitrile composition comprises less than 1 wt % of impurities. In some embodiments, the acetonitrile composition comprises less than 0.5 wt % of impurities. In some embodiments, the acetonitrile composition comprises less than 0.1 wt % of impurities. In some embodiments, the wt% of impurities is measured by gas chromatography.

In some embodiments, the acetonitrile composition comprises an absorbance of less than 0.1 absorbance units (AU) at a wavelength of 200 nm. In some embodiments, the acetonitrile composition comprises an absorbance of less than 0.05 absorbance units (AU) at a wavelength of 200 nm.

In some embodiments, the acetonitrile composition comprises an absorbance of less than 0.1 AU at a wavelength of 210 nm. In some embodiments, the acetonitrile composition comprises an absorbance of less than 0.03 AU at a wavelength of 210 nm.

In some embodiments, the acetonitrile composition comprises an absorbance of less than 0.05 AU at a wavelength of 220 nm. In some embodiments, the acetonitrile composition comprises an absorbance of less than 0.02 AU at a wavelength of 220 nm.

In some embodiments, the acetonitrile composition comprises an absorbance of less than 0.05 AU at a wavelength of 230 nm. In some embodiments, the acetonitrile composition comprises an absorbance of less than 0.01 AU at a wavelength of 230 nm.

In some embodiments, the acetonitrile composition comprises an absorbance of less than 0.01 AU at a wavelength of 240 nm. In some embodiments, the acetonitrile composition comprises an absorbance of less than 0.005 AU at a wavelength of 240 nm.

In some embodiments, the acetonitrile composition comprises an absorbance of less than 0.01 AU at a wavelength of 260 nm. In some embodiments, the acetonitrile composition comprises an absorbance of less than 0.005 AU at a wavelength of 260 nm.

In some embodiments, the acetonitrile composition comprises an absorbance of less than 0.01 AU at a wavelength of 280 nm. In some embodiments, the acetonitrile composition comprises an absorbance of less than 0.005 AU at a wavelength of 280 nm.

In some embodiments, the acetonitrile composition comprises an absorbance of less than 0.01 AU at a wavelength of 400 nm. In some embodiments, the acetonitrile composition comprises an absorbance of less than 0.005 AU at a wavelength of 400 nm.

In some embodiments, the acetonitrile composition comprises no more than 20 ppm water. In some embodiments, the acetonitrile composition comprises no more than 10 ppm water. In some embodiments, the acetonitrile composition is anhydrous acetonitrile composition.

In some embodiments, the biologically produced acetonitrile precursor is produced by fermentation. In some embodiments, the biologically produced acetonitrile precursor is produced by bacterial fermentation. In some embodiments, the biologically produced acetonitrile precursor is produced by aerobic bacterial fermentation.

In some embodiments, the purifying the biologically produced acetonitrile precursor comprises liquid-liquid extraction, distillation, or a combination thereof. In some embodiments, the liquid-liquid extraction comprises contacting the biologically produced acetonitrile precursor with an organic solvent. In some embodiments, the organic solvent is ethyl acetate, butyl acetate, diethyl ether, dichloromethane, toluene, chloroform, methyl tert-butyl ether, toluene, chloroform, hexane, benzene, acetone, or a combination thereof.

In some embodiments, the biologically produced acetonitrile precursor is acetic acid. In some embodiments, the biologically produced acetonitrile precursor is glacial acetic acid.

In some embodiments, the catalyst is a heterogeneous catalyst. In some embodiments, the catalyst is aluminum oxide, titanium dioxide, zirconium dioxide, tungsten oxide, or combinations thereof. In some embodiments, the catalyst is a transition metal oxide or a combination of transition metal oxides. In some embodiments, the transition metal oxide is zirconium dioxide, tungsten oxide, or a combination thereof.

In some embodiments, the reacting comprises heating to a temperature of no more than 350° C. In some embodiments, the reacting comprises heating to a temperature of from about 250° C. to about 330° C.

In some embodiments, the nitrogen source is ammonia. In some embodiments, the nitrogen source is anhydrous ammonia.

In some embodiments, the reacting comprises a pressure of from about 0.8 atm to about 5 atm. In some embodiments, the reacting comprise a pressure of from about 1 atm to about 4 atm.

In some embodiments, the purifying the crude acetonitrile comprises distillation, oxidation, polishing, or a combination thereof. In some embodiments, the distillation removes excess nitrogen source. In some embodiments, the excess nitrogen source is recycled in (c).

In some embodiments, the purifying comprises use of an oxidant. In some embodiments, the oxidant is a Lewis acid. In some embodiments, the oxidant is potassium permanganate, ozone, or potassium superoxide. In some embodiments, the oxidant is potassium permanganate.

In some embodiments, the purifying the crude acetonitrile is completed in acidic, neutral, or basic conditions. In some embodiments, the purifying the crude acetonitrile is completed in basic conditions.

In some embodiments, the polishing of the crude acetonitrile comprises contacting the crude acetonitrile with a zeolite, ion-exchange resin, activated alumina, activated carbon, or a combination thereof. In some embodiments, the polishing of the crude acetonitrile comprises contacting the crude acetonitrile with silica, calcium sulfate, molecular sieves, a zeolite, alumina, or magnesium sulfate.

In some embodiments, the method produces acetonitrile composition in a quantity of at least 100 L. In some embodiments, the method produces acetonitrile composition in a quantity of at least 150 L.

In some embodiments, the acetonitrile composition is pharmaceutical grade acetonitrile composition. In some embodiments, the acetonitrile composition is oligonucleotide synthesis grade acetonitrile composition.

In some embodiments, the method does not produce acrylonitrile. In some embodiments, the method does not comprise use of an acetonitrile precursor derived from a fossil fuel.

In some embodiments, the acetonitrile composition comprises a reduction in carbon intensity (CI) as compared to an acetonitrile composition produced using the SOHIO process. In some embodiments, the acetonitrile composition comprises an about 90% reduction in CI as compared to an acetonitrile composition produced using the SOHIO process.

Provided herein, in some embodiments, is a composition comprising acetonitrile provided by any of the methods described herein.

Provided herein, in some embodiments, is a composition comprising acetonitrile, wherein the composition comprises at least 98 wt % acetonitrile derived from a biologically produced precursor, less than 10 ppm water, and does not comprise acetonitrile derived from a fossil fuel source.

In some embodiments, the composition comprises at least 99 wt % acetonitrile derived from a biologically produced precursor. In some embodiments, the composition comprises at least 99.5 wt % acetonitrile derived from a biologically produced precursor. In some embodiments, the composition comprises at least 99.9 wt % acetonitrile derived from a biologically produced precursor.

In some embodiments, the composition comprises an absorbance of less than 0.1 absorbance units (AU) at a wavelength of 200 nm. In some embodiments, the composition comprises an absorbance of less than 0.05 absorbance units (AU) at a wavelength of 200 nm.

In some embodiments, the composition comprises an absorbance of less than 0.1 AU at a wavelength of 210 nm. In some embodiments, the composition comprises an absorbance of less than 0.03 AU at a wavelength of 210 nm.

In some embodiments, the composition comprises an absorbance of less than 0.05 AU at a wavelength of 220 nm. In some embodiments, the composition comprises an absorbance of less than 0.02 AU at a wavelength of 220 nm.

In some embodiments, the composition comprises an absorbance of less than 0.05 AU at a wavelength of 230 nm. In some embodiments, the composition comprises an absorbance of less than 0.01 AU at a wavelength of 230 nm.

In some embodiments, the composition comprises an absorbance of less than 0.01 AU at a wavelength of 240 nm. In some embodiments, the composition comprises an absorbance of less than 0.005 AU at a wavelength of 240 nm.

In some embodiments, the composition comprises an absorbance of less than 0.01 AU at a wavelength of 260 nm. In some embodiments, the composition comprises an absorbance of less than 0.005 AU at a wavelength of 260 nm.

In some embodiments, the composition comprises an absorbance of less than 0.01 AU at a wavelength of 280 nm. In some embodiments, the composition comprises an absorbance of less than 0.005 AU at a wavelength of 280 nm.

In some embodiments, the composition comprises an absorbance of less than 0.01 AU at a wavelength of 400 nm. In some embodiments, the composition comprises an absorbance of less than 0.005 AU at a wavelength of 400 nm.

In some embodiments, the composition comprises anhydrous acetonitrile.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows an illustrative method for preparing acetonitrile as described herein. In FIG. 1, in some embodiments, each of (102), (103), (104), and (105) may be required. In some instances, (102) may be optional. In some instances, (103) may be optional. In some instances, (104) may be optional. In some instances, (105) may be optional. In some instances, (107) may be optional. In some instances, (112) may be optional.

FIG. 2 shows an illustrative method for preparing acetonitrile as described herein. In FIG. 2, each of (203), (204), and (205) may be required. In some instances, (203) may be optional. In some instances, (204) may be optional. In some instances, (205) may be optional. In some instances, (211) may be optional.

FIG. 3 shows an exemplary reaction mechanism for preparation of acetonitrile from acetic acid.

FIG. 4 shows an illustrative method for purifying acetonitrile as described herein. In FIG. 4, (401) may be optional. In FIG. 4, (402) may be optional. In FIG. 4, in some embodiments, both (401) and (402) may be required.

FIG. 5 shows an illustrative procedure for production of acetic acid from fermentation of ethanol.

FIG. 6 shows an illustrative procedure for purifying acetic acid as described herein.

### DETAILED DESCRIPTION

### Certain Definitions

As used herein and in the appended claims, the singular forms “a,” “and,” and “the” include plural referents unless the context clearly dictates otherwise. Thus, for example, reference to “an agent” includes a plurality of such agents and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

As used herein, “pure” or “purity” refer to a substance that is free from or comprises less than a given level or amount of other substances or impurities. For example, the substance can have e.g., 5 wt% or less (e.g., 4 wt %, 3 wt %, 2.5 wt %, 2 wt%, 1.5 wt % 1 wt %, 0.5 wt %, 0.1 wt %, or 0.05 wt %) of total impurities or related substances. “Percent pure” or “percent purity” as used herein refers to a substance comprising less than a given percentage (e.g., wt%, vol%, mol%) of impurities or related substances. For instance, a substance that is 95% pure may refer to the substance comprising less than 5% of impurities or related substances.

Methods for determining purity may include, but are not limited to, gas chromatography.

### Methods of Making Acetonitrile

Acetonitrile is a solvent often used in analytical chemistry and many manufacturing processes. It is historically produced from the propylene ammoxidation process (also known as the SOHIO process). In the SOHIO process, propylene, ammonia, and oxygen are reacted at high temperature to produce acrylonitrile, where acetonitrile is produced as a byproduct. There are several disadvantages to the currently used SOHIO process in that (1) it requires the use of fossil fuel sources (e.g., propylene), (2) acetonitrile is a byproduct of the SOHIO process, (3) the process is very carbon intensive, (4) the reaction produces toxic side products, including hydrogen cyanide, (5) the reaction requires the use of oxygen which represents an explosive hazard, and (6) high purity grade acetonitrile is difficult to achieve using the SOHIO process.

Provided herein, in some embodiments, are methods of making acetonitrile (or compositions comprising acetonitrile), also referred to herein as “bio-based acetonitrile”. The methods provided herein are alternatives to the standard propylene ammoxidation process, which allow for preparation of acetonitrile at high levels of purity (e.g., oligonucleotide synthesis quality or pharma grade quality). In some embodiments, the methods herein provide for the use of alternative feedstocks, such as ethanol and sugars, which obviate the need for fossil fuel based sources (e.g., propylene). For instance, the methods herein provide for the production of (e.g., high purity) acetonitrile by use of 100% bio-based feedstocks. The methods provided herein may be less carbon intensive as a result, allowing for a more ecologically sustainable method for preparation of acetonitrile. Additionally, the methods herein are decoupled from production of other reagents (e.g., acrylonitrile) and provide for direct preparation of acetonitrile that does not require the use of explosive hazards or result in the release of toxic byproduct, such as hydrogen cyanide.

The acetonitrile (e.g., bio-based acetonitrile) composition provided herein may comprise a reduction in carbon intensity (CI) compared to acetonitrile produced using traditional fossil-fuel based methods, such as the SOHIO process. In some embodiments, the acetonitrile (e.g., bio-based acetonitrile) composition comprises an at least 50%, 60%, 70%, 80%, 85%, 86%, 87%, 88%, 89%, 90%, 91%, 92%, 93%, 94%, 95%, 96%, 97%, 98%, 99%, 99.5%, or 100% reduction in CI as compared to an acetonitrile composition produced using traditional fossil-fuel based methods, such as the SOHIO process. In some embodiments, the acetonitrile (e.g., bio-based acetonitrile) composition comprises an at least 90% reduction in CI as compared to an acetonitrile composition produced using traditional fossil-fuel based methods, such as the SOHIO process. In some embodiments, the acetonitrile (e.g., bio-based acetonitrile) composition comprises an about 90% reduction in CI as compared an acetonitrile produced composition using the SOHIO process. In some embodiments, CI is measured using the GREET 2023 model (e.g., as described at http://greet.anl.gov/).

Carbon Intensity (CI), as described herein, can refer to a measure of carbon dioxide and other greenhouse gases per unit of activity (e.g., manufacture of acetonitrile). In some embodiments, CI is measured using the The Greenhouse gases, Regulated Emissions, and Energy use in Technologies Model (GREET), which uses input data related to the lifecycle of various fuels and transportation systems and outputs the calculated CI, determined by assessing the total greenhouse gas emissions produced per unit of activity (e.g., as described at http://greet.anl. gov/). In some embodiments, CI is measured using the GREET 2023 model as described at http://greet.anl.gov/.

In some embodiments, the methods provided herein comprise providing an acetonitrile precursor. In some embodiments, the acetonitrile precursor is acetic acid, an acetate salt (e.g., sodium acetate, potassium acetate, calcium acetate, ammonium acetate, zinc acetate, copper acetate, manganese acetate, iron acetate, magnesium acetate, aluminum acetate, lithium acetate, barium acetate, cobalt acetate, lead acetate, nickel acetate, silver acetate, chromium acetate, chromium acetate, cadmium acetate, or strontium acetate), acetamide, acetaldehyde, acetic anhydride, a substituted or unsubstituted C₂ carbonyl (e.g., ketone or carboxylic acid), or a combination thereof. In some embodiments, the acetonitrile precursor is an acetate salt. In some embodiments, the acetonitrile precursor is acetamide. In some embodiments, the acetonitrile precursor is acetaldehyde. In some embodiments, the acetonitrile precursor is acetic anhydride. In specific embodiments, the acetonitrile precursor is acetic acid.

In some embodiments, the acetonitrile precursor is produced from a biological source (e.g., a biologically produced acetonitrile precursor). The biologically produced acetonitrile precursor may be produced by any suitable method.

In some embodiments, the acetonitrile precursor is provided via catalytic oxidation. In some embodiments, the acetonitrile precursor is acetic acid. In some embodiments, the acetonitrile precursor is acetic acid and is provided via catalytic oxidation of ethanol to acetic acid. In some embodiments, catalytic oxidation of ethanol to acetic acid comprises conversion of ethanol to acetaldehyde. In some embodiments, the catalytic oxidation of ethanol to acetic acid comprises reaction of acetaldehyde to form the acetic acid.

In some embodiments, the methods provided herein incorporate no fossil-derived material into the acetonitrile. For instance, the methods provided herein do not comprise the use of propylene, propane, or any combination thereof.

In some embodiments, the methods comprise use of an acetonitrile precursor comprising at least 80 wt % (e.g., at least 90 wt %, 95 wt %, 99 wt %) of a biologically produced acetonitrile precursor (e.g., acetic acid). In some embodiments, the methods comprise use of an acetonitrile precursor comprising at least 80 wt % of a biologically produced acetonitrile precursor (e.g., acetic acid). In some embodiments, the methods comprise use of an acetonitrile precursor comprising at least 95 wt % of a biologically produced acetonitrile precursor (e.g., acetic acid). In some embodiments, the methods comprise use of an acetonitrile precursor comprising 100 wt % of a biologically produced acetonitrile precursor (e.g., acetic acid), such as biologically produced via the methods described herein.

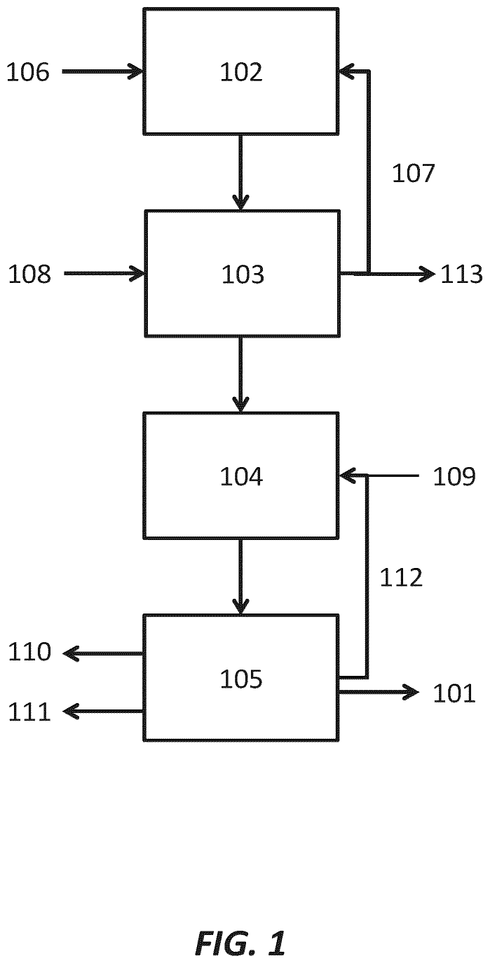

In some embodiments, the biologically produced acetonitrile precursor (e.g., acetic acid) is produced by fermentation. In some embodiments, the biologically produced acetonitrile precursor (e.g., acetic acid) is produced by fermentation, such as described in Example 1 and Example 2, elsewhere herein. In some embodiments, the biologically produced acetonitrile precursor (e.g., acetic acid) is produced by fermentation, such as shown in FIG. 1 (102). In some embodiments, the fermentation is aerobic fermentation. In some embodiments, the fermentation is anaerobic fermentation. In some embodiments, the biologically produced acetonitrile precursor is produced by fermentation of ethanol. In some embodiments, the fermentation is bacterial fermentation. In some embodiments, the fermentation is fungal fermentation. In some embodiments, the fermentation is anaerobic bacterial fermentation. In some embodiments, the fermentation is aerobic bacterial fermentation.

In some embodiments, the fermentation is conducted as a cyclical production process.

In some embodiments, the cyclical production process is a batch process. In some embodiments, the batch process comprises a charge phase. In some embodiments, the batch process comprises a fermentation phase. In some embodiments, the batch process comprises a discharge phase. In some embodiments, the batch process comprises a charge phase, a fermentation phase, a discharge phase, or a combination thereof.

In some embodiments, the cyclical production process is a fed-batch process. In some embodiments, the fed-batch process comprises a charge phase. In some embodiments, the fed-batch process comprises a fermentation start phase. In some embodiments, the fed-batch process comprises a substrate feed phase. In some embodiments, the fed-batch process comprises a fermentation finish phase. In some embodiments, the fed-batch process comprises a discharge phase. In some embodiments, the fed-batch process comprises a charge phase, a fermentation start phase, a substrate feed phase, a fermentation finish phase, a discharge phase, or a combination thereof.

In some embodiments, the fermentation is conducted as a continuous process. In some embodiments, the continuous process comprises a continuous inflow of mash balanced with simultaneous withdrawal of fermentation broth such that the fermentation reaches a “steady state.” In some instances, the fermentation comprises use of a feedstock comprising sugar. The sugar may be a sugar from corn, sugar beets, sugar cane, or a combination thereof.

In some embodiments, the acetonitrile precursor (e.g., acetic acid) is impure. In some embodiments, the impure acetonitrile precursor (e.g., acetic acid) is provided in a solution comprising at most 40 wt % (e.g., at most 35 wt%, 30 wt %, 25 wt %, 20 wt %, 15 wt %, 10 wt %, or 5 wt %) of the acetonitrile precursor (e.g., acetic acid).

In some embodiments, the impurities in the impure acetonitrile precursor may comprise any organic or inorganic impurities.

In some embodiments, the acetonitrile precursor (e.g., acetic acid) is pure. In some embodiments, the pure acetonitrile precursor (e.g., acetic acid) is provided in a solution comprising at least 80 wt % (e.g., at least 85 wt %, 90 wt %, 95 wt %, 97 wt %, 98 wt %, 99 wt %, or 99.5 wt %) of the acetonitrile precursor (e.g., acetic acid).

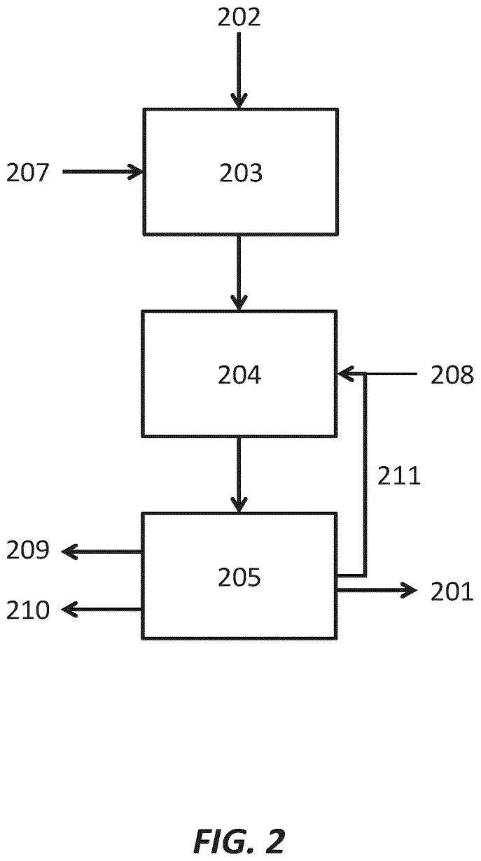

In some embodiments, the methods provided herein comprise purifying the (e.g., biologically produced) acetonitrile precursor, such as shown in FIG. 1 (103) and FIG. 2 (203).

In some embodiments, the purifying may comprise a plurality of steps.

The purifying may comprise filtration. In some embodiments, filtration removes rejected biomass. In some embodiments, filtration removes cells (e.g., from bacterial fermentation). In some embodiments, filtration comprises hollow fiber tangential flow filtration, spiral wound tangential flow filtration, flat sheet tangential flow filtration, dynamic filtration, dead-end filtration, direct flow filtration, ceramic membrane filtration, crossflow filtration, ultrafiltration, depth filtration, or nanofiltration. In some embodiments, filtration comprises spiral wound tangential flow filtration. In some embodiments, filtration comprises hollow fiber tangential flow filtration. In some embodiments, filtration comprises flat sheet tangential flow filtration. In some embodiments, filtration comprises dynamic filtration. In some embodiments, filtration comprises dead-end filtration. In some embodiments, filtration comprises dead-end filtration. In some embodiments, filtration comprises direct flow filtration. In some embodiments, filtration comprises ceramic membrane filtration. In some embodiments, filtration comprises crossflow filtration. In some embodiments, filtration comprises ultrafiltration. In some embodiments, filtration comprises nanofiltration. In some embodiments, filtration comprises depth filtration.

In some embodiments, filtration comprises use of any suitable pore size. In some embodiments, filtration comprises use of filter with pore size of at least 0.05 um (e.g., at least 0.1 um, 0.2 um, 0.3 μm, 0.5 μm, 0.6 μm, 0.8 μm, 1 μm, 2 μm, 3 μm, 5 μm, 6 μm, 8 μm, 10 μm, 12 μm, 13 μm, 15 μm, 16 μm, 18 μm, or at least 20 μm). In some embodiments, filtration comprises uses of a filter with pore size of at most 50 μm (e.g., at most 45 μm, 40 μm, 35 μm, 30 μm, 25 μm, 20 μm, 15 μm, 10 μm, 8 μm, 6 μm, 5 μm, 4 μm, 2 μm, or at most 1 μm). In some embodiments, filtration comprises use of a filter with pore size of from about 0.05 μm to about 50 μm, from about 0.1 μm to about 20 μm, from about 0.5 μm to about 20 μm, from about 1 μm to about 20 μm, from about 0.1 μm to about 10 μm, from about 0.1 μm to about 1 μm, or from about 0.1 μm to about 0.8 μm. In some embodiments, filtration comprises use of a filter with pore size of about 0.1 μm, 0.2 μm, 0.3 μm, 0.4 μm, 0.5 μm, 0.6 μm, 0.7 μm, 0.8 μm, 0.9 μm, or 1 μm. In some embodiments, the pore size is 0.2 μm. In some embodiments, the pore size is 0.45 μm. In some embodiments, the pore size is 0.5 μm.

In some embodiments, filtration comprises use of spiral wound tangential flow filtration with pore size of about 0.5 μm. In some embodiments, filtration comprises use of spiral wound tangential flow filtration with pore size of about 0.2 μm. In some embodiments, filtration comprises use of spiral wound tangential flow filtration with pore size of about 0.45 μm.

In some embodiments, filtration comprises filtering the (e.g., biologically produced) acetonitrile precursor using a plurality of filters, such as filter with pore size described herein. In some embodiments, filtration comprises filtering the (e.g., biologically produced) acetonitrile precursor using 2 filters. In some embodiments, filtration comprises filtering the (e.g., biologically produced) acetonitrile precursor using 3 filters. In some embodiments, filtration comprises filtering the (e.g., biologically produced) acetonitrile precursor using 4 filters. In some embodiments, the plurality of filters (e.g., 2 or more filters) comprise differing pore sizes, such as pores sizes described elsewhere herein. For instance, one filter may comprise a pore size of about 0.5 μm, while the second filter may comprise a pore size of less than 0.5 μm. In some embodiments, the first filter may comprise a greater pore size than the second filter.

In some embodiments, filtration membrane materials comprise polyethersulfone (PES), polytetrafluoroethylene (PTFE or Teflon), polyvinylidene fluoride (PVDF), polypropylene (PP), cellulose acetate, polysulfone, nylon, polyethylene, polyvinyl alcohol (PVA), or ceramic tubular filter materials. In some embodiments, filtration membrane materials comprise polyethersulfone (PES) or poltetrafluoroethylene (PTFE or Teflon).

In some embodiments, filtration comprises use of a filter aid. In some embodiments, filtration is completed absent use of a filter aid. In instances where a filter aid is used, a filter aid may comprise diatomaceous earth, activated charcoal, cellulose, perlite, activated alumina, bentonite, silica gel, fuller's earth, or cotton.

In some embodiments, filtration of the (e.g., biologically produced) acetonitrile precursor provides a clarified solution. In some instances, when the acetonitrile precursor is acetic acid, the clarified solution comprises acetic acid, ethanol, and water.

The clarified solution may comprise at least 5 wt % (e.g., at least 7 wt %, 8 wt %, 10 wt %, 12 wt%, 14 wt %, 16 wt %, 18 wt %, or at least 20 wt %) of the (e.g., biologically produced) acetonitrile precursor. The clarified solution may comprise at most 50 wt % (e.g., at most 45 wt %, 40 wt %, 35 wt%, 30 wt %, 28 wt %, 26 wt %, 25 wt %, 24 wt %, 22 wt %, or 20 wt %) of the (e.g., biologically produced) acetonitrile precursor.

The purifying may comprise liquid-liquid extraction, distillation, or a combination thereof. The combination of liquid-liquid extraction and distillation may be referred to as hybrid-extraction-distillation, such as described in Example 1 and Example 2.

The purifying may comprise liquid-liquid extraction. In some embodiments, the liquid-liquid extraction comprises contacting the (e.g., biologically produced) acetonitrile precursor with an organic solvent (FIG. 1 (108), FIG. 2 (207)). The organic solvent may be any suitable organic solvent in order to purify the acetonitrile precursor. In some embodiments, the organic solvent is ethyl acetate, butyl acetate, diethyl ether, dichloromethane, toluene, chloroform, methyl tert-butyl ether, toluene, chloroform, hexane, benzene, acetone, or a combination thereof. In some embodiments, the organic solvent comprises ethyl acetate. In some embodiments, the organic solvent comprises butyl acetate. In some embodiments, the organic solvent is diethyl ether. In some embodiments, the organic solvent is dichloromethane. In some embodiments, the organic solvent is toluene. In some embodiments, the organic solvent is chloroform. In some embodiments, the organic solvent is methyl tert-butyl ether. In some embodiments, the organic solvent is toluene. In some embodiments, the organic solvent is chloroform. In some embodiments, the organic solvent is hexane. In some embodiments, the organic solvent is benzene. In some embodiments, the organic solvent is acetone. In some embodiments, liquid-liquid extraction is performed once, twice, or three or more times during purification of the acetonitrile precursor. In some embodiments, liquid-liquid extraction is not performed.

In some embodiments, the extraction comprises agitation. In some embodiments, the agitation comprises vertical agitation, horizontal agitation, or a combination thereof. In some embodiments, the agitation comprises vertical (axial) agitation. In some embodiments, the agitation comprises horizontal agitation. In some embodiments, the agitation does not comprise horizontal agitation. In some embodiments, the agitation does not comprise vertical agitation.

In some embodiments, purifying comprises solid phase extraction (SPE). In some embodiments, the SPE is normal phase SPE, reverse phase SPE, or mixed-mode SPE. In some embodiments, the adsorbent used in SPE is silica gel, alumina, C18, C8, C2, Oasis HLB, or Strata X. In some embodiments, the adsorbent used in SPE is silica gel or alumina. In some embodiments, the adsorbent used in C18, C8, or C2. In some embodiments, the adsorbent used in Oasis HLB or Strata X.

The purifying may comprise distillation. Distillation may be used to remove one or more organic solvents present in the (e.g., biologically produced) acetonitrile precursor. The one or more organic solvents may comprise ethanol (e.g., from the fermentation process described elsewhere herein) or an organic solvent described hereinabove. In some embodiments, distillation is performed once, twice, or three or more times during purification of the acetonitrile precursor. In some embodiments, distillation is not performed.

The purifying may comprise stripping. Stripping may be used to remove one or more organic solvents present in the (e.g., biologically produced) acetonitrile precursor. The one or more organic solvents may comprise ethanol (e.g., from the fermentation process described elsewhere herein) or an organic solvent described hereinabove. In some embodiments, stripping is performed once, twice, or three or more times during purification of the acetonitrile precursor. In some embodiments, stripping is not performed.

In some embodiments, excess water may be removed from the (e.g., biologically produced) acetonitrile precursor.

In some embodiments, residual water and organic solvent removed from the acetonitrile precursor during purification is recycled. In some embodiments, water and ethanol (e.g., from the fermentation described hereinabove) is recycled and used in further fermentation. In some embodiments, recycling of water and/or ethanol is shown in FIG. 1 (107). In some embodiments, recycling of water and/or ethanol (shown in FIG. 1 (107)) is optional. In some cases the water and/or ethanol is removed as a waste stream (FIG. 1 (113)).

In some embodiments, the product of purifying the (e.g., biologically produced) acetonitrile precursor is a purified (e.g., biologically produced) acetonitrile precursor. In some embodiments, the (e.g., biologically produced) acetonitrile precursor is at least 85% (e.g., at least 87%, 88%, 90%, 92%, 94%, 95%, 96%, 97%, 98%, 99%, 99.5%, 99.7%, 99.9%, or 99.99%) pure. In some embodiments, the (e.g., biologically produced) acetonitrile precursor is from about 85% to about 99.99% pure, from about 90% to about 99.9% pure, from about 90% to about 99.5% pure, from about 95% to about 99.9% pure, or from about 95% to about 99% pure. In some embodiments, the purified acetonitrile precursor is anhydrous. In some instances, the anhydrous purified acetonitrile precursor comprises no more than 30 ppm of water.

In some embodiments, the (e.g., biologically produced) acetonitrile precursor comprises no more than 15 wt % (e.g., no more than 13 wt %, 12 wt %, 10 wt %, 8 wt %, 6 wt %, 5 wt %, 4 wt %, 3 wt%, 2 wt %, 1 wt %, 0.5 wt %, 0.3 wt %, 0.1 wt %, or 0.01 wt %) of impurities, such as after purification. In some embodiments, the (e.g., biologically produced) acetonitrile precursor comprises no more than 0.01 wt % to 15 wt % of impurities, from about 0.1 wt % to 10 wt % of impurities from 0.5 wt % to 10 wt% of impurities from 0.1 wt % to 5 wt % of impurities, or from 1 wt % to 5 wt % of impurities.

In specific embodiments, the (e.g., biologically produced) acetonitrile precursor is acetic acid. In some embodiments, purifying the acetonitrile provides glacial acetic acid. In some embodiments, the acetic acid is at least 85% (e.g., at least 87%, 88%, 90%, 92%, 94%, 95%, 96%, 97%, 98%, 99%, 99.5%, 99.7%, 99.9%, or 99.99%) pure. In some embodiments, the acetic acid is from about 85% to about 99.99% pure, from about 90% to about 99.9% pure, from about 90% to about 99.5% pure, from about 95% to about 99.9% pure, or from about 95% to about 99% pure. In some embodiments, the purified acetic acid (e.g., glacial acetic acid) is anhydrous.

In some embodiments, impurities in acetic acid comprise any organic or inorganic impurities other than acetic acid.

In some embodiments, the (e.g., purified) acetic acid comprises no more than 15 wt % (e.g., no more than 13 wt %, 12 wt %, 10 wt %, 8 wt %, 6 wt %, 5 wt %, 4 wt %, 3 wt %, 2 wt %, 1 wt %, 0.5 wt %, 0.3 wt %, 0.1 wt %, or 0.01 wt %) of impurities, such as after purification. In some embodiments, the (e.g., purified) acetic acid comprises no more than 0.01 wt % to 15 wt % of impurities, from about 0.1 wt% to 10 wt % of impurities from 0.5 wt % to 10 wt % of impurities from 0.1 wt % to 5 wt % of impurities, or from 1 wt % to 5 wt % of impurities.

In some embodiments, the methods provided herein comprise reacting the (e.g., biologically produced) acetonitrile precursor with a nitrogen source to provide (e.g., crude) acetonitrile.

The reaction of the acetonitrile precursor with the nitrogen source to provide acetonitrile may be performed using any suitable method. In some embodiments, the reaction is nitrilation.

In some embodiments, nitrilation includes a combination of (1) ammonolysis to produce an amide and (2) amide dehydration.

In some embodiments, the reacting of the acetonitrile precursor with the nitrogen source does not produce hydrogen cyanide.

In some embodiments, the reacting of the acetonitrile precursor with the nitrogen source does not produce acrylonitrile.

In some embodiments, the nitrogen source is ammonia, an ammonium salt (e.g., ammonium bicarbonate, ammonium carbonate, ammonium carbamate, ammonium chloride, ammonium acetate, or ammonium sulfate), or ammonium hydroxide. In some embodiments, the nitrogen source is ammonia. In some embodiments, the nitrogen source is an ammonium salt (e.g., ammonium bicarbonate, ammonium carbonate, ammonium carbamate, ammonium chloride, ammonium acetate, or ammonium sulfate). In some embodiments, the nitrogen source is ammonium hydroxide. In some embodiments, the ammonia is provided as a recycled (unreacted) product of subsequent steps (e.g., as shown in FIG. 1 (112), FIG. 2 (211)). In some embodiments, the recycling of ammonia from subsequent steps (e.g., as shown in FIG. 1 (112), FIG. 2 (211)) is optional.

In some embodiments, the reaction is completed at elevated temperature. In some embodiments, the reaction is completed at a temperature of at least 200° C. (e.g., at least 210° C., 220° C., 230° C., 240° C., 250° C., 260° C., 270° C., 280° C., 290° C., or at least 300° C.). In some embodiments, the reaction is completed at a temperature of no more than 350° C. (e.g., no more than 340° C., 330° C., 320° C., 310° C., or 300° C.). In some embodiments, the reaction is completed at a temperature of no more than 450° C. (e.g., no more than 425° C., 400° C., 380° C., 370° C., or 360° C.). In some embodiments, the reaction is completed at a temperature of from about 200° C. to about 450° C., from about 200° C. to about 400° C., from about 200° C. to about 350° C., from about 220° C. to about 250° C., from about 250° C. to about 330° C., from about 290° C. to about 340° C., or from about 290° C. to about 320° C. In some embodiments, the reaction is completed at a temperature of about 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., 320° C., 325° C., or about 330° C.

In some embodiments, reactants are pre-heated before reaction, such as to prevent formation of unfavorable side products. In some embodiments, pre-heating comprises heating to a temperature of at least 180° C. (e.g., at least 190° C., 200° C., 210° C., 220° C., 230° C., 240° C., 250 ° C., or at least 260° C.).

As described herein in Example 1 and Example 2, side reactions may become more pronounced at higher temperatures and reactor fouling may occur at lower temperatures due to incomplete reaction.

In some embodiments, the reaction is completed at standard pressure or elevated pressures. In some embodiments, the reaction is completed at a combination of standard pressures and elevated pressures. In some embodiments, the reaction is completed at standard pressure. In some embodiments, the reaction is completed at elevated pressure. In some embodiments, the reaction is completed at a pressure of at least 0.8 atm (e.g., at least 0.85 atm, 0.9 atm, 0.95 atm, 0.1 atm, 0.2 atm, 0.4 atm, 0.5 atm, 1 atm, 1.25 atm, 1.5 atm, 1.75 atm, 2 atm, 3 atm, 4 atm, 5 atm, 6 atm, 7 atm, 8 atm, 9 atm, or at least 10 atm). In some embodiments, the reaction is completed at a pressure of at most 10 atm (e.g., at most 9 atm, 8 atm, 7 atm, 6 atm, 5 atm, 4 atm, 3 atm, 2 atm, or 1 atm). In some embodiments, the reaction is completed at a pressure of from about 1 atm to about 10 atm, from about 1 atm to about 8 atm, from about 1 atm to about 6 atm, from about 1 atm to about 5 atm, from about 1atm to about 3 atm, or from about 1 atm to about 4 atm. In some embodiments, the reaction is completed at a temperature of about 1 atm, 1.2 atm, 1.4 atm, 1.6 atm, 1.8 atm, 2 atm, 2.2 atm, 2.4 atm, 2.6 atm, 2.8 atm, 3 atm, 3.2 atm, 3.4 atm, 3.6 atm, 3.8 atm, 4 atm, 4.2 atm, 4.4 atm, 4.6 atm, 4.8 atm, or about 5 atm. In some instances, the pressure is absolute pressure.

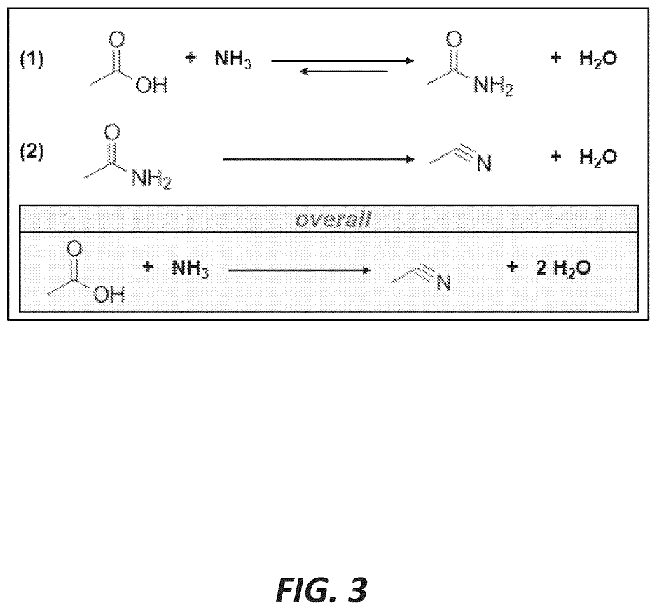

In some embodiments, the reaction comprises contacting (e.g., glacial) acetic acid as provided or produced in the preceding steps with ammonia to produce (e.g., crude) acetonitrile, as depicted in FIG. 3.

In some embodiments, the reaction comprises use of a catalyst. In some embodiments, the catalyst is a heterogeneous catalyst. In other embodiments, the catalyst is a homogeneous catalyst. In some embodiments, the catalyst is a metal oxide. In some embodiments, the catalyst is a transition metal oxide. In some embodiments, the catalyst is aluminum oxide, titanium oxide, zirconium oxide, tungsten oxide, or a combination thereof. In some embodiments, the catalyst is aluminum oxide. In some embodiments, the catalyst is titanium oxide. In some embodiments, the catalyst is zirconium oxide. In some embodiments, the catalyst is tungsten oxide.

The catalyst may be contained in a fixed bed reactor, a trickle-bed reactor, a moving bed reactor, a rotating bed reactor, a slurry reactor, or a fluidized bed reactor.

In some embodiments, the reacting provided herein produces no more than 30 wt % of organic products other than acetonitrile. In some embodiments, the reacting provided herein produces no more than 20 wt % of organic products other than acetonitrile. In some embodiments, the reacting provided herein produces no more than 15 wt % of organic products other than acetonitrile. In some embodiments, the reacting provided herein produces no more than 10 wt % of organic products other than acetonitrile. In some embodiments, the reacting provided herein produces no more than 9 wt % of organic products other than acetonitrile. In some embodiments, the reacting provided herein produces no more than 8 wt % of organic products other than acetonitrile. In some embodiments, the reacting provided herein produces no more than 7 wt % of organic products other than acetonitrile. In some embodiments, the reacting provided herein produces no more than 5 wt % of organic products other than acetonitrile.

In some embodiments, the methods herein comprise purifying the (e.g., crude) acetonitrile (e.g., as depicted in FIG. 1 (105), FIG. 2 (205)).

In some embodiments, the methods provided herein comprise purifying (e.g., crude) acetonitrile that is produced by any suitable method, such as propylene ammoxidation.

In some embodiments, purifying the (e.g., crude) acetonitrile comprises distillation, oxidation, polishing, pervaporation, hybrid extraction-distillation, extractive distillation, salting out, sugaring out, freezing out, or a combination thereof. In some embodiments, purifying the (e.g., crude) acetonitrile comprises distillation. In some embodiments, purifying the (e.g., crude) acetonitrile comprises oxidation. In some embodiments, purifying the (e.g., crude) acetonitrile comprises polishing. In some embodiments, purifying the (e.g., crude) acetonitrile comprises pervaporation. In some embodiments, purifying the (e.g., crude) acetonitrile comprises hybrid extraction-distillation. In some embodiments, purifying the (e.g., crude) acetonitrile comprises extractive distillation. In some embodiments, purifying the (e.g., crude) acetonitrile comprises salting out. In some embodiments, purifying the (e.g., crude) acetonitrile comprises sugaring out. In some embodiments, purifying the (e.g., crude) acetonitrile comprises freezing out.

In some embodiments, purifying the (e.g., crude) acetonitrile comprises distillation. In some embodiments, the distillation removes unreacted nitrogen source from the (e.g., crude) acetonitrile. In some embodiments, the distillation removes at least 85 wt % (e.g., at least 90 wt %, 92 wt %, 94 wt %, 95 wt %, 96 wt %, 97 wt %, 98 wt %, 99 wt %, 99.5 wt %, 99.9 wt %, or at least 99.99 wt %) of the unreacted nitrogen source from the (e.g., crude) acetonitrile. In some embodiments, the distillation removes from about 85 wt % to about 100 wt %, from about 90 wt % to about 100 wt %, from about 94 wt % to about 100 wt %, or from about 97 wt % to about 100 wt % of the unreacted nitrogen source from the (e.g., crude) acetonitrile.

In some embodiments, the nitrogen source removed from the (e.g., crude) acetonitrile may be recycled. The recycled nitrogen source may be used in the reaction step with the acetonitrile precursor, as described elsewhere herein (FIG. 1 (112), FIG. 2 (211)). In some embodiments, the nitrogen source removed from the (e.g., crude) acetonitrile may be removed and stored. The nitrogen source may be removed and stored by use of e.g., cryogenic distillation or an entrainer.

In some embodiments, purifying the (e.g., crude) acetonitrile comprises distillation. In some embodiments, the distillation removes unreacted ammonia from the (e.g., crude) acetonitrile. In some embodiments, the distillation removes at least 85 wt % (e.g., at least 90 wt %, 92 wt%, 94 wt %, 95 wt %, 96 wt %, 97 wt %, 98 wt %, 99 wt %, 99.5 wt %, 99.9 wt %, or at least 99.99 wt %) of ammonia from the (e.g., crude) acetonitrile. In some embodiments, the distillation removes from about 85 wt % to about 100 wt %, from about 90 wt % to about 100 wt %, from about 94 wt % to about 100 wt %, or from about 97 wt % to about 100 wt % of the ammonia from the (e.g., crude) acetonitrile.

In some embodiments, the ammonia removed from the (e.g., crude) acetonitrile may be recycled. The recycled ammonia may be used in the reaction step with the acetonitrile precursor, as described elsewhere herein (FIG. 1 (112), FIG. 2 (211)). In some embodiments, the ammonia removed from the (e.g., crude) acetonitrile may be removed and stored. The ammonia may be removed and stored by use of e.g., cryogenic distillation or an entrainer.

In some embodiments, distillation removes volatile impurities from the (e.g., crude) acetonitrile. The volatile impurities may include ammonia, carbon dioxide, acetone, or a combination thereof. In some embodiments, distillation removes carbon dioxide from the (e.g., crude) acetonitrile. In some embodiments, distillation removes acetone from the (e.g., crude) acetonitrile (FIG. 1 (111), FIG. 2 (210)).

In some instances, impurities present in the (e.g., crude) acetonitrile include acetonitrile and carbon dioxide. These impurities may be a result of ketonization of acetic acid.

In some embodiments, distillation comprises pressure swing distillation, extractive distillation, azeotropic distillation, heterogeneous azeotropic distillation, or vacuum distillation. In some embodiments, the distillation comprises pressure swing distillation. In some embodiments, the distillation comprises extractive distillation. In some embodiments, the distillation comprises azeotropic distillation. In some embodiments, the distillation comprises heterogeneous azeotropic distillation. In some embodiments, the distillation comprises vacuum distillation.

In some embodiments, impurities in the (e.g., crude) acetonitrile include other nitriles (e.g., 3-methyl 2-butene nitrile and propionitrile), aromatics, acetone, carbon dioxide, ammonium salts, water, or a combination thereof. In some instances, the unsaturated nitriles may be present at a concentration of about 100 ppm. In some instances, the total aromatics may be present at a concentration of about 20 ppm. Further purification may be required to meet certain specifications, such as for oligonucleotide grade quality, as described elsewhere herein.

In some embodiments, purifying the (e.g., crude) acetonitrile comprises oxidation, e.g., oxidation of impurities. In some cases, the oxidation of impurities allows for more facile removal of the impurities from the (e.g., crude) acetonitrile enabling further purification.

In some embodiments, purifying the (e.g., crude) acetonitrile comprises use of an oxidant. In some embodiments, the oxidant is a Lewis acid. In some embodiments, the oxidant is potassium permanganate, ozone, potassium superoxide, potassium persulfate, hydrogen peroxide, sodium hypochlorite, sodium persulfate, nitric acid, sulfuric acid, chlorine dioxide, or lead dioxide. In some embodiments, the oxidant is potassium permanganate, ozone, or potassium superoxide. In some embodiments, the oxidant is potassium permanganate. In some embodiments, the oxidant is ozone. In some embodiments, the oxidant is potassium superoxide.

In some embodiments, purifying the (e.g., crude) acetonitrile comprises use of acidic, basic, or neutral conditions. In some embodiments, purifying the (e.g., crude) acetonitrile comprises use of acidic conditions. In some embodiments, purifying the (e.g., crude) acetonitrile comprises use of neutral conditions. In some embodiments, purifying the (e.g., crude) acetonitrile comprises use of basic conditions.

In some embodiments, the oxidation is completed in acidic, basic, or neutral conditions. In some embodiments, the oxidation is completed in acidic conditions. Acidic conditions may be provided by addition of hydrochloric acid, hydrobromic acid, sulfuric acid, nitric acid, perchloric acid, or any other suitable acid. In some embodiments, the oxidation is completed in neutral conditions. In some embodiments, neutral conditions comprise use of no acid or base. In some embodiments, neutral conditions comprise use of a buffer. In some embodiments, the oxidation is completed in basic conditions. Basic conditions may be provided by addition of a base, such as sodium hydroxide, potassium hydroxide, calcium hydroxide, magnesium hydroxide, or lithium hydroxide.

In some embodiments, the purifying comprises polishing. In some embodiments, purifying the (e.g., crude) acetonitrile (polishing) comprises contacting the (e.g., crude) acetonitrile with a zeolite, ion-exchange resin, activated carbon, silica, calcium sulfate, molecular sieves, alumina, activated alumina, magnesium sulfate, or a combination thereof. In some embodiments, purifying the (e.g., crude) acetonitrile (polishing) comprises contacting the (e.g., crude) acetonitrile with at least two of a zeolite, ion-exchange resin, activated carbon, silica, calcium sulfate, molecular sieves, alumina, activated alumina, and magnesium sulfate.

In some embodiments, purifying the (e.g., crude) acetonitrile (polishing) comprises first contacting the (e.g., crude) acetonitrile with a first polishing material followed by contacting the (e.g., crude) acetonitrile with a second polishing material. In some embodiments, purifying the (e.g., crude) acetonitrile (polishing) comprises first contacting the (e.g., crude) acetonitrile with a first polishing material. In some embodiments, purifying the (e.g., crude) acetonitrile (polishing) comprises first contacting the (e.g., crude) acetonitrile with a second polishing material. In some embodiments, the polishing material is a zeolite, ion-exchange resin, activated carbon, silica, calcium sulfate, molecular sieves, alumina, activated alumina, magnesium sulfate, or a combination thereof. In some embodiments, the first polishing material is a zeolite, ion-exchange resin, activated carbon, silica, calcium sulfate, molecular sieves, alumina, activated alumina, or magnesium sulfate. In some embodiments, the second polishing material is a zeolite, ion-exchange resin, activated carbon, silica, calcium sulfate, molecular sieves, alumina, activated alumina, or magnesium sulfate. In some embodiments, the first polishing material and the second polishing material are the same. In some embodiments, the first polishing material and the second polishing material are different.

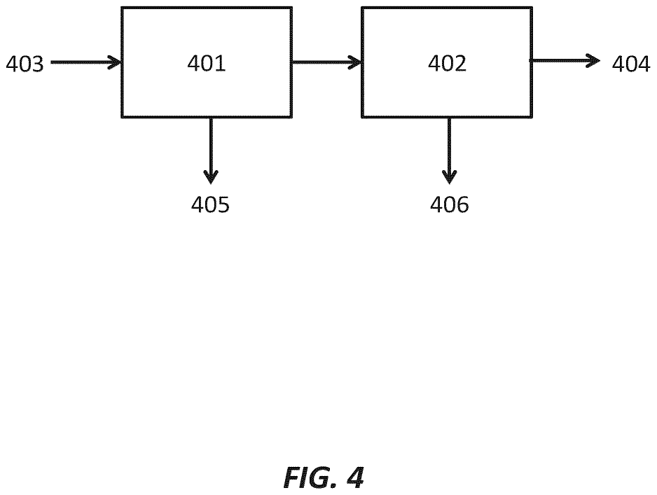

In some embodiments, purifying the (e.g., crude) acetonitrile (polishing) comprises first contacting the (e.g., crude) acetonitrile with a zeolite, ion-exchange resin, activated carbon, silica, calcium sulfate, molecular sieves, alumina, activated alumina, or magnesium sulfate (FIG. 4 (401)), followed by second, contacting the (e.g., crude) acetonitrile (polishing) with a (e.g., second portion of a) zeolite, ion-exchange resin, activated carbon, silica, calcium sulfate, molecular sieves, alumina, activated alumina, and magnesium sulfate (FIG. 4 (402)). In some embodiments, purifying the (e.g., crude) acetonitrile (polishing) comprises first contacting the (e.g., crude) acetonitrile with a first zeolite, ion-exchange resin, activated carbon, silica, calcium sulfate, molecular sieves, alumina, activated alumina, magnesium sulfate, or a combination thereof (FIG. 4 (401)). In some embodiments, purifying the (e.g., crude) acetonitrile (polishing) comprises contacting the (e.g., crude) acetonitrile with a second zeolite, ion-exchange resin, activated carbon, silica, calcium sulfate, molecular sieves, alumina, activated alumina, magnesium sulfate, or a combination thereof (FIG. 4 (402)). In some embodiments, the zeolite, ion-exchange resin, activated carbon, silica, calcium sulfate, molecular sieves, alumina, activated alumina, magnesium sulfate, or a combination thereof in 401 is the same as the zeolite, ion-exchange resin, activated carbon, silica, calcium sulfate, molecular sieves, alumina, activated alumina, magnesium sulfate, or a combination thereof in 402. In some embodiments, the zeolite, ion-exchange resin, activated carbon, silica, calcium sulfate, molecular sieves, alumina, activated alumina, magnesium sulfate, or a combination thereof in 401 is different than the zeolite, ion-exchange resin, activated carbon, silica, calcium sulfate, molecular sieves, alumina, activated alumina, magnesium sulfate, or a combination thereof in 402.

In some embodiments, purifying the (e.g., crude) acetonitrile (polishing) comprises contacting the (e.g., crude) acetonitrile with a zeolite. In some embodiments, purifying the (e.g., crude) acetonitrile comprises contacting the (e.g., crude) acetonitrile (polishing) with an ion-exchange resin. In some embodiments, purifying the (e.g., crude) acetonitrile (polishing) comprises contacting the (e.g., crude) acetonitrile with activated carbon. In some embodiments, purifying the (e.g., crude) acetonitrile (polishing) comprises contacting the (e.g., crude) acetonitrile with silica. In some embodiments, purifying the (e.g., crude) acetonitrile (polishing) comprises contacting the (e.g., crude) acetonitrile with calcium sulfate. In some embodiments, purifying (polishing) the (e.g., crude) acetonitrile comprises contacting (polishing) the (e.g., crude) acetonitrile with molecular sieves. In some embodiments, purifying the (e.g., crude) acetonitrile (polishing) comprises contacting the (e.g., crude) acetonitrile with alumina. In some embodiments, purifying the (e.g., crude) acetonitrile (polishing) comprises contacting the (e.g., crude) acetonitrile with activated alumina. In some embodiments, purifying the (e.g., crude) acetonitrile (polishing) comprises contacting the (e.g., crude) acetonitrile with magnesium sulfate.

In some embodiments, purifying the (e.g., crude) acetonitrile (polishing) comprises contacting the (e.g., crude) acetonitrile (403) with activated carbon (401), such as to remove impurity aromatic compounds (405) such as depicted in FIG. 4. In some embodiments, purifying the (e.g., crude) acetonitrile (polishing) comprises contacting the (e.g., crude) acetonitrile with activated alumina (402), such as to remove trace water (406) such as depicted in FIG. 4. The contacting with e.g., alumina or activated carbon (or any other purification reagents described herein) may be completed in packed absorption columns.

In some embodiments, the scheme shown in FIG. 4 is comprised in FIG. 1 (105) or FIG. 2 (205).

In some embodiments the resulting acetonitrile after purification of the (e.g., crude) acetonitrile is purified acetonitrile (FIG. 1 (101), FIG. 2 (201)).

The acetonitrile prepared by the methods provided herein may be prepared at both lab-and industrial-scales. In some embodiments, the method produces acetonitrile (compositions) in a quantity of at least 100 mL (e.g., at least 500 mL, 1 L, 5 L, 10 L, 15 L, 20 L, or 25 L). In other embodiments, the method produces acetonitrile (compositions) in a quantity of at least 100 L (e.g., at least 150 L, 200 L, 250 L, 300 L, 350 L, 400 L, 450 L, 500 L, 600 L, 700 L, 800 L, 900 L, 1000 L, 2500 L, 5000 L, 7500 L, or 10000 L).

In some embodiments, the methods provided herein may prepare acetonitrile (compositions) at a rate of at least 0.5 kg/h (e.g., at least 0.75 kg/h, 1 kg/h, 1.5 kg/h, 2 kg/h, 2.5 kg/h, 3 kg/h, 4 kg/h, 5 kg/h, 6 kg/h, 7 kg/h, 8 kg/h, 9 kg/h, 10 kg/h, or more). In some embodiments, the methods herein prepare acetonitrile (compositions) at a rate of at least 5 kg/h. In some embodiments, the methods herein prepare acetonitrile at a rate of at least 6 kg/h.

### Purified Bio-Acetonitrile

In some embodiments, provided herein are acetonitrile compositions prepared by any of the methods provided herein. In some embodiments, the acetonitrile compositions provided herein, prepared by the methods provided herein, may be, for example, oligonucleotide synthesis grade, ACS grade, HPLC grade, or LC/MS grade acetonitrile compositions.

In some embodiments, the acetonitrile composition is at least 90% pure. In some embodiments, the acetonitrile composition is at least 95% pure. In some embodiments, the acetonitrile composition is at least 97.5% pure. In some embodiments, the acetonitrile composition is at least 99% (e.g., at least 99.1%, 99.2%, 99.3%, 99.4%, 99.5%, 99.6%, 99.7%, 99.8%, or 99.9%) pure. In some embodiments, the acetonitrile composition is at least 99.6% pure. In some embodiments, the acetonitrile composition is at least 99.7% pure. In some embodiments, the acetonitrile composition is at least 99.8% pure. In some embodiments, the acetonitrile composition is at least 99.9% pure. In some embodiments, the acetonitrile composition is from about 90% to about 99.99% pure, about 95% to about 99.99% pure, about 97% to about 99.99% pure, about 98% to about 99.99% pure, or about 99% to about 99.99% pure.

The purity of the acetonitrile compositions described herein may be measured by gas chromatography (GC) coupled with mass spectrometry (MS), flame ionization detectors (FID), thermal conductivity detectors (TCD), or a combination thereof. In some instances, the purity of the acetonitrile composition is measured by GC-MS. In some instances, the purity of the acetonitrile composition is measured by GC-MS/FID/TCD. Absorbance of the acetonitrile compositions as described herein may be measured using UV-Vis spectroscopy, such as using a path length of 1 cm. The acetonitrile compositions provided herein (e.g., the identity of the acetonitrile) may be measured by use of Fourier Transform Infrared Spectroscopy (FTIR) or nuclear magnetic resonance spectroscopy (NMR). The water content of the acetonitrile compositions provided herein may be measured using Karl Fischer titration.

In some embodiments, the acetonitrile composition comprises less than 10 wt % of impurities (e.g., organic or inorganic impurities or water). In some embodiments, the acetonitrile composition comprises less than 5 wt % of impurities. In some embodiments, the acetonitrile composition comprises less than 1 wt % (e.g., less than 0.9 wt %, 0.8 wt %, 0.7 wt %, 0.6 wt %, 0.5 wt %, 0.4 wt %, 0.3 wt %, 0.2 wt %, or 0.1 wt %) of impurities. In some embodiments, the acetonitrile composition comprises no more than 0.5 wt % of impurities. In some embodiments, the acetonitrile composition comprises no more than 0.4 wt % of impurities. In some embodiments, the acetonitrile composition comprises no more than 0.3 wt % of impurities. In some embodiments, the acetonitrile composition comprises no more than 0.2 wt % of impurities. In some embodiments, the acetonitrile composition comprises no more than 0.1 wt % of impurities. In some embodiments, the acetonitrile composition comprises from 0 wt % to about 10 wt % of impurities, from about 0.01 wt % to about 5 wt% of impurities, from about 0.01 wt % to about 3 wt % of impurities, from about 0.01 wt % to about 2 wt % of impurities, or from 0.01 wt % to about 1 wt % of impurities. In some embodiments, the acetonitrile composition comprises 0.01 wt %, 0.02 wt %, 0.05 wt %, 0.1 wt %, 0.15 wt %, 0.2 wt %, 0.25 wt %, 0.3 wt %, 0.35 wt %, 0.4 wt %, 0.45 wt %, 0.5 wt %, 0.75 wt %, 1 wt %, 1.5 wt %, 2 wt %, 2.5 wt %, 3 wt %, 3.5 wt %, 4 wt %, 4.5 wt %, or 5 wt % of impurities.

The amount (e.g., wt%) of impurities in the acetonitrile composition may be measured by gas chromatography.

In some embodiments, the acetonitrile impurities may comprise any organic or inorganic impurities (e.g., organic or inorganic impurities produced by the methods provided herein).

The acetonitrile identity may be confirmed by infrared spectroscopy or nuclear magnetic resonance (NMR) spectroscopy.

In some embodiments, the density of the acetonitrile composition at 20° C. may be about 0.774 g/mL. In some embodiments, the density of the acetonitrile composition at 20° C. may be about 0.775 g/mL. In some embodiments, the density of the acetonitrile composition at 20° C. may be about 0.776 g/mL. In some embodiments, the density of the acetonitrile composition at 20° C. may be about 0.778 g/mL. In some embodiments, the density of the acetonitrile composition at 20° C. may be about 0.779 g/mL. In some embodiments, the density of the acetonitrile composition at 20° C. may be about 0.780 g/mL. In some embodiments, the density of the acetonitrile composition at 20° C. may be about 0.781 g/mL. In some embodiments, the density of the acetonitrile composition at 20° C. may be about 0.782 g/mL. In some embodiments, the density of the acetonitrile composition at 20° C. may be about 0.783 g/mL. In some embodiments, the density of the acetonitrile composition at 20° C. may be about 0.784 g/mL. In some embodiments, the density of the acetonitrile composition at 20° C. may be about 0.785 g/mL. In some embodiments, the density of the acetonitrile composition at 20° C. may be about 0.786 g/mL. In some embodiments, the density of the acetonitrile composition at 20° C. may be at least 0.774 g/mL (e.g., at least 0.776 g/mL, 0.777 g/mL, 0.778 g/mL, or 0.780 g/mL). In some embodiments, the density of the acetonitrile composition at 20° C. may be at most 0.785 g/mL (e.g., at most 0.784 g/mL, 0.783 g/mL, 0.782 g/mL, 0.781 g/mL, or 0.780 g/mL). In some embodiments, the density of the acetonitrile composition at 20° C. may be from about 0.778 g/mL to about 0.786 g/mL. In some embodiments, the density of the acetonitrile composition at 20° C. may be from about 0.780 g/mL to about 0.783 g/mL.

In some embodiments, the refractive index at 20° C. of the acetonitrile composition is about 1.340. In some embodiments, the refractive index at 20° C. of the acetonitrile composition is about 1.341. In some embodiments, the refractive index at 20° C. of the acetonitrile composition is about 1.342. In some embodiments, the refractive index at 20° C. of the acetonitrile composition is about 1.343. In some embodiments, the refractive index at 20° C. of the acetonitrile composition is about 1.344. In some embodiments, the refractive index at 20° C. of the acetonitrile composition is about 1.345. In some embodiments, the refractive index at 20° C. of the acetonitrile composition is about 1.346. In some embodiments, the refractive index at 20° C. of the acetonitrile composition is about 1.347. In some embodiments, the refractive index at 20° C. of the acetonitrile composition is about 1.348. In some embodiments, the refractive index at 20° C. of the acetonitrile composition is about 1.349. In some embodiments, the refractive index at 20° C. of the acetonitrile composition is no more than 1.349. In some embodiments, the refractive index at 20° C. of the acetonitrile composition is no more than 1.346. In some embodiments, the refractive index at 20° C. of the acetonitrile composition is from about 1.341 to about 1.349. In some embodiments, the refractive index at 20° C. of the acetonitrile composition is from about 1.343 to about 1.346.

In some embodiments, the acetonitrile composition comprises less than 1000 ppm (e.g., less than 900 ppm, 800 ppm, 700 ppm, 600 ppm, 500 ppm, 400 ppm, 300 ppm, 200 ppm, or less than 100 ppm) of water. In some embodiments, the acetonitrile composition comprises less than 100 ppm (e.g., less than 90 ppm, 80 ppm, 70 ppm, 60 ppm, 50 ppm, 40 ppm, 30 ppm, 20 ppm, 10 ppm, or less than 5 ppm) of water. In some embodiments, the acetonitrile composition comprises less than 50 ppm of water. In some embodiments, the acetonitrile composition comprises less than 20 ppm of water. In some embodiments, the acetonitrile composition comprises less than 10 ppm of water. In some embodiments, the acetonitrile composition comprises less than 5 ppm of water. In some embodiments, the acetonitrile composition comprises less than 1 ppm of water. In some embodiments, the acetonitrile composition comprises from about 1 ppm to about 50 ppm of water, from about 1 ppm to about 25 ppm of water, from about 1 ppm to about 20 ppm of water, from about 1 ppm to about 10 ppm of water, or from about 1 ppm to about 5 ppm of water.

Water content in the acetonitrile composition may be measured by Karl Fischer titration.

In some embodiments, the acetonitrile composition is anhydrous.

In some embodiments, the acetonitrile composition comprises an acidity (as CH3COOH) of less than 0.01% (e.g., less than 0.009%, 0.008%, 0.007%, 0.006%, 0.005%, 0.004%, 0.003%, 0.002%, or 0.001%). In some embodiments, the acetonitrile composition comprises an acidity (as CH₃COOH) of less than 0.001% (e.g., less than 0.0009%, 0.0008%, 0.0007%, 0.0006%, 0.0005%, 0.0004%, 0.0003%, 0.0002%, or 0.0001%). In some embodiments, the acetonitrile composition comprises an acidity (as CH₃COOH) of less than 0.001%. In some embodiments, the acetonitrile composition comprises an acidity (as CH₃COOH) of less than 0.0005%. In some embodiments, the acetonitrile composition comprises an acidity (as CH₃COOH) of from about 0.0001% to about 0.05%, from about 0.0005% to about 0.005%, or from about 0.0008% to about 0.002%.

In some embodiments, the acetonitrile composition comprises less than 20 μeq/g (e.g., less than 18 μeq/g, 16 μeq/g, 15 μeq/g, 14 μeq/g, 13 μeq/g, 12 μeq/g, 11 μeq/g, 10 μeq/g, 9 μeq/g, 8 peq/g, 7 μeq/g, 6 μeq/g, 5 μeq/g, 4 μeq/g, 3 μeq/g, 2 μeq/g, 1 μeq/g, 0.5 μeq/g, or less than 0.1 μeq/g) of titratable acid. In some embodiments, the acetonitrile composition comprises less than 15 μeq/g of titratable acid. In some embodiments, the acetonitrile composition comprises less than 10 μeq/g of titratable acid. In some embodiments, the acetonitrile composition comprises less than 8 μeq/g of titratable acid. In some embodiments, the acetonitrile composition comprises less than 5 μeq/g of titratable acid. In some embodiments, the acetonitrile composition comprises from about 0.1 μeq/g to about 20 μeq/g, about 0.1 μeq/g to about 15 μeq/g, about 0.1 μeq/g to about 10 μeq/g, or from about 0.1 μeq/g to about 5 μeq/g of titratable acid.

In some embodiments, the acetonitrile composition comprises less than 10 μeq/g (e.g., less than 8 μeq/g, 6 μeq/g, 5 μeq/g, 4 μeq/g, 3 μeq/g, 2 μeq/g, 1 μeq/g, 0.9 μeq/g, 0.8 μeq/g, 0.7 μeq/g, 0.6 μeq/g, 0.5 μeq/g, 0.4 μeq/g, 0.3 μeq/g, 0.2 μeq/g, 0.1 μeq/g, 0.05 μeq/g, or less than 0.01 μeq/g) of titratable base. In some embodiments, the acetonitrile composition comprises less than 1 μeq/g of titratable base. In some embodiments, the acetonitrile composition comprises less than 0.8 μeq/g of titratable base. In some embodiments, the acetonitrile composition comprises less than 0.6 μeq/g of titratable base. In some embodiments, the acetonitrile composition comprises less than 0.3 μeq/g of titratable base. In some embodiments, the acetonitrile composition comprises from about 0.01 μeq/g to about 10 μeq/g, about 0.01 μeq/g to about 1 μeq/g, about 0.01 μeq/g to about 0.6 μeq/g, or from about 0.01 μeq/g to about 0.03 μeq/g of titratable base.

In some embodiments, the acetonitrile composition comprises a residue after evaporation of less than 0.01% (e.g., less than less than 0.009%, 0.008%, 0.007%, 0.006%, 0.005%, 0.004%, 0.003%, 0.002%, or 0.001%). In some embodiments, the acetonitrile composition comprises a residue after evaporation of less than 0.001% (e.g., less than 0.0009%, 0.0008%, 0.0007%, 0.0006%, 0.0005%, 0.0004%, 0.0003%, 0.0002%, or 0.0001%). In some embodiments, the acetonitrile composition comprises a residue after evaporation of less than 0.0005%. In some embodiments, the acetonitrile composition comprises a residue after evaporation of from 0.0001% to about 0.01%, from about 0.0005% to about 0.005%, from about 0.0005% to about 0.002%, or from about 0.0008% to about 0.002%.

In some embodiments, the purity of the acetonitrile composition may be measured by absorbance. The absorbance may be measured using UV-vis spectroscopy.

In some embodiments, the acetonitrile composition comprises an absorbance at a wavelength of 195 nm of less than 1 AU (e.g., less than 0.9 AU, 0.8 AU, 0.7 AU, 0.6 AU, 0.5 AU, 0.4 AU, 0.3 AU, 0.2 AU, or less than 0.1 AU). In some embodiments, the acetonitrile composition comprises an absorbance at a wavelength of 195 nm of less than 0.9 AU. In some embodiments, the acetonitrile composition comprises an absorbance at a wavelength of 195 nm of less than 0.8 AU. In some embodiments, the acetonitrile composition comprises an absorbance at a wavelength of 195 nm of less than 0.6 AU. In some embodiments, the acetonitrile composition comprises an absorbance at a wavelength of 195 nm of less than 0.5 AU. In some embodiments, the acetonitrile composition comprises an absorbance at a wavelength of 195 nm of from about 0.1 AU to about 1 AU, from about 0.2 AU to about 0.8 AU, from about 0.3 AU to about 0.9 AU, or from about 0.4 AU to about 1 AU.

In some embodiments, the acetonitrile composition comprises an absorbance at a wavelength of 195 nm of less than 0.3 AU (e.g., less than 0.28 AU, 0.25 AU, 0.24 AU, 0.22 AU, 0.2 AU, 0.18 AU, 0.16 AU, 0.15 AU, 0.14 AU, 0.12 AU, 0.1 AU, 0.08 AU, 0.06 AU, or less than 0.05 AU). In some embodiments, the acetonitrile composition comprises an absorbance at a wavelength of 195 nm of less than 0.2 AU. In some embodiments, the acetonitrile composition comprises an absorbance at a wavelength of 195 nm of less than 0.15 AU. In some embodiments, the acetonitrile composition comprises an absorbance at a wavelength of 195 nm of less than 0.1 AU. In some embodiments, the acetonitrile composition comprises an absorbance at a wavelength of 195 nm of from 0.05 AU to about 0.3 AU, from about 0.05 AU to about 0.025 AU, from about 0.05 AU to about 0.02 AU, from about 0.1 AU to about 0.3 AU, or from about 0.1 AU to about 0.2 AU.

In some embodiments, the acetonitrile composition comprises an absorbance at a wavelength of 200 nm of less than 0.2 AU (e.g., less than 0.19 AU, 0.18 AU, 0.17 AU, 0.16 AU, 0.15 AU, 0.14 AU, 0.13 AU, 0.12 AU, 0.11 AU, 0.10 AU, 0.09 AU, 0.08 AU, 0.07 AU, 0.06 AU, 0.05 AU, 0.04 AU, 0.03 AU, 0.02 AU, or less than 0.01 AU). In some embodiments, the acetonitrile composition comprises an absorbance at a wavelength of 200 nm of less than 0.15 AU. In some embodiments, the acetonitrile composition comprises an absorbance at a wavelength of 200 nm of less than 0.1 AU. In some embodiments, the acetonitrile composition comprises an absorbance at a wavelength of 200 nm of less than 0.09 AU. In some embodiments, the acetonitrile composition comprises an absorbance at a wavelength of 200 nm of less than 0.08 AU. In some embodiments, the acetonitrile composition comprises an absorbance at a wavelength of 200 nm of less than 0.07 AU. In some embodiments, the acetonitrile composition comprises an absorbance at a wavelength of 200 nm of less than 0.06 AU. In some embodiments, the acetonitrile composition comprises an absorbance at a wavelength of 200 nm of less than 0.05 AU. In some embodiments, the acetonitrile composition comprises an absorbance at a wavelength of 200 nm of less than 0.04 AU. In some embodiments, the acetonitrile composition comprises an absorbance at a wavelength of 200 nm of less than 0.03 AU. In some embodiments, the acetonitrile composition comprises an absorbance at a wavelength of 200 nm of from 0.01 AU to about 0.1 AU, from about 0.01 AU to about 0.09 AU, from about 0.01 AU to about 0.08 AU, from about 0.01 AU to about 0.07 AU, or from about 0.01 AU to about 0.05 AU.

In some embodiments, the acetonitrile composition comprises an absorbance at a wavelength of 210 nm of less than 0.2 AU (e.g., less than 0.18 AU, 0.17 AU, 0.16 AU, 0.15 AU, 0.14 AU, 0.13 AU, 0.12 AU, 0.11 AU, 0.10 AU, 0.09 AU, 0.08 AU, 0.07 AU, 0.06 AU, 0.05 AU, 0.04 AU, 0.03 AU, 0.02 AU, 0.01, or less than 0.005 AU). In some embodiments, the acetonitrile composition comprises an absorbance at a wavelength of 210 nm of less than 0.15 AU. In some embodiments, the acetonitrile composition comprises an absorbance at a wavelength of 210 nm of less than 0.1 AU. In some embodiments, the acetonitrile composition comprises an absorbance at a wavelength of 210 nm of less than 0.08 AU. In some embodiments, the acetonitrile composition comprises an absorbance at a wavelength of 210 nm of less than 0.06 AU. In some embodiments, the acetonitrile composition comprises an absorbance at a wavelength of 210 nm of less than 0.05 AU. In some embodiments, the acetonitrile composition comprises an absorbance at a wavelength of 210 nm of less than 0.04 AU. In some embodiments, the acetonitrile composition comprises an absorbance at a wavelength of 210 nm of less than 0.03 AU. In some embodiments, the acetonitrile composition comprises an absorbance at a wavelength of 210 nm of less than 0.02 AU. In some embodiments, the acetonitrile composition comprises an absorbance at a wavelength of 210 nm of less than 0.01 AU. In some embodiments, the acetonitrile composition comprises an absorbance at a wavelength of 210 nm of from 0.005 AU to about 0.1 AU, from about 0.005 AU to about 0.08 AU, from about 0.005 AU to about 0.04 AU, from about 0.005 AU to about 0.03 AU, or from about 0.01 AU to about 0.03 AU.

In some embodiments, the acetonitrile composition comprises an absorbance at a wavelength of 220 nm of less than 0.2 AU (e.g., less than 0.18 AU, 0.17 AU, 0.16 AU, 0.15 AU, 0.14 AU, 0.13 AU, 0.12 AU, 0.11 AU, 0.10 AU, 0.09 AU, 0.08 AU, 0.07 AU, 0.06 AU, 0.05 AU, 0.04 AU, 0.03 AU, 0.02 AU, 0.01, or less than 0.005 AU). In some embodiments, the acetonitrile composition comprises an absorbance at a wavelength of 220 nm of less than 0.15 AU. In some embodiments, the acetonitrile composition comprises an absorbance at a wavelength of 220 nm of less than 0.1 AU. In some embodiments, the acetonitrile composition comprises an absorbance at a wavelength of 220 nm of less than 0.08 AU. In some embodiments, the acetonitrile composition comprises an absorbance at a wavelength of 220 nm of less than 0.06 AU. In some embodiments, the acetonitrile composition comprises an absorbance at a wavelength of 220 nm of less than 0.05 AU. In some embodiments, the acetonitrile composition comprises an absorbance at a wavelength of 220 nm of less than 0.04 AU. In some embodiments, the acetonitrile composition comprises an absorbance at a wavelength of 220 nm of less than 0.03 AU. In some embodiments, the acetonitrile composition comprises an absorbance at a wavelength of 220 nm of less than 0.02 AU. In some embodiments, the acetonitrile composition comprises an absorbance at a wavelength of 220 nm of less than 0.01 AU. In some embodiments, the acetonitrile composition comprises an absorbance at a wavelength of 220 nm of from 0.005 AU to about 0.1 AU, from about 0.005 AU to about 0.08 AU, from about 0.005 AU to about 0.04 AU, from about 0.005 AU to about 0.03 AU, or from about 0.01 AU to about 0.03 AU.

In some embodiments, the acetonitrile composition comprises an absorbance at a wavelength of 230 nm of less than 0.15 AU (e.g., less than 0.14 AU, 0.12 AU, 0.10 AU, 0.09 AU, 0.08 AU, 0.07 AU, 0.06 AU, 0.05 AU, 0.04 AU, 0.03 AU, 0.02 AU, 0.01, 0.005 AU, or less than 0.001 AU). In some embodiments, the acetonitrile composition comprises an absorbance at a wavelength of 230 nm of less than 0.1 AU. In some embodiments, the acetonitrile composition comprises an absorbance at a wavelength of 230 nm of less than 0.05 AU. In some embodiments, the acetonitrile composition comprises an absorbance at a wavelength of 230 nm of less than 0.02 AU. In some embodiments, the acetonitrile composition comprises an absorbance at a wavelength of 230 nm of less than 0.01 AU. In some embodiments, the acetonitrile composition comprises an absorbance at a wavelength of 230 nm of less than 0.005 AU. In some embodiments, the acetonitrile composition comprises an absorbance at a wavelength of 230 nm of less than 0.003 AU. In some embodiments, the acetonitrile composition comprises an absorbance at a wavelength of 230 nm of less than 0.001 AU. In some embodiments, the acetonitrile composition comprises an absorbance at a wavelength of 230nm of from 0.001 AU to about 0.1 AU, from about 0.001 AU to about 0.08 AU, from about 0.001 AU to about 0.02 AU, from about 0.001 AU to about 0.01 AU, or from about 0.005 AU to about 0.01 AU.

In some embodiments, the acetonitrile composition comprises an absorbance at a wavelength of 240 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 240 nm of less than 0.015 AU. In some embodiments, the acetonitrile composition comprises an absorbance at a wavelength of 240 nm of less than 0.01 AU. In some embodiments, the acetonitrile composition comprises an absorbance at a wavelength of 240 nm of less than 0.009 AU. In some embodiments, the acetonitrile composition comprises an absorbance at a wavelength of 240 nm of less than 0.008 AU. In some embodiments, the acetonitrile composition comprises an absorbance at a wavelength of 240 nm of less than 0.007 AU. In some embodiments, the acetonitrile composition comprises an absorbance at a wavelength of 240 nm of less than 0.006 AU. In some embodiments, the acetonitrile composition comprises an absorbance at a wavelength of 240 nm of less than 0.005 AU. In some embodiments, the acetonitrile composition comprises an absorbance at a wavelength of 240 nm of less than 0.004 AU. In some embodiments, the acetonitrile composition comprises an absorbance at a wavelength of 240 nm of less than 0.003 AU. In some embodiments, the acetonitrile composition comprises an absorbance at a wavelength of 240 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition comprises an absorbance at a wavelength of 260 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 260 nm of less than 0.015 AU. In some embodiments, the acetonitrile composition comprises an absorbance at a wavelength of 260 nm of less than 0.01 AU. In some embodiments, the acetonitrile composition comprises an absorbance at a wavelength of 260 nm of less than 0.009 AU. In some embodiments, the acetonitrile composition comprises an absorbance at a wavelength of 260 nm of less than 0.008 AU. In some embodiments, the acetonitrile composition comprises an absorbance at a wavelength of 260 nm of less than 0.007 AU. In some embodiments, the acetonitrile composition comprises an absorbance at a wavelength of 260 nm of less than 0.006 AU. In some embodiments, the acetonitrile composition comprises an absorbance at a wavelength of 260 nm of less than 0.005 AU. In some embodiments, the acetonitrile composition comprises an absorbance at a wavelength of 260 nm of less than 0.004 AU. In some embodiments, the acetonitrile composition comprises an absorbance at a wavelength of 260 nm of less than 0.003 AU. In some embodiments, the acetonitrile composition comprises an absorbance at a wavelength of 260 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition comprises an absorbance at a wavelength of 280 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 280 nm of less than 0.015 AU. In some embodiments, the acetonitrile composition comprises an absorbance at a wavelength of 280 nm of less than 0.01 AU. In some embodiments, the acetonitrile composition comprises an absorbance at a wavelength of 280 nm of less than 0.009 AU. In some embodiments, the acetonitrile composition comprises an absorbance at a wavelength of 280 nm of less than 0.008 AU. In some embodiments, the acetonitrile composition comprises an absorbance at a wavelength of 280 nm of less than 0.007 AU. In some embodiments, the acetonitrile composition comprises an absorbance at a wavelength of 280 nm of less than 0.006 AU. In some embodiments, the acetonitrile composition comprises an absorbance at a wavelength of 280 nm of less than 0.005 AU. In some embodiments, the acetonitrile composition comprises an absorbance at a wavelength of 280 nm of less than 0.004 AU. In some embodiments, the acetonitrile composition comprises an absorbance at a wavelength of 280 nm of less than 0.003 AU. In some embodiments, the acetonitrile composition comprises an absorbance at a wavelength of 280 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition comprises an absorbance at a wavelength of 400 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 400 nm of less than 0.015 AU. In some embodiments, the acetonitrile composition comprises an absorbance at a wavelength of 400 nm of less than 0.01 AU. In some embodiments, the acetonitrile composition comprises an absorbance at a wavelength of 400 nm of less than 0.009 AU. In some embodiments, the acetonitrile composition comprises an absorbance at a wavelength of 400 nm of less than 0.008 AU. In some embodiments, the acetonitrile composition comprises an absorbance at a wavelength of 400 nm of less than 0.007 AU. In some embodiments, the acetonitrile composition comprises an absorbance at a wavelength of 400 nm of less than 0.006 AU. In some embodiments, the acetonitrile composition comprises an absorbance at a wavelength of 400 nm of less than 0.005 AU. In some embodiments, the acetonitrile composition comprises an absorbance at a wavelength of 400 nm of less than 0.004 AU. In some embodiments, the acetonitrile composition comprises an absorbance at a wavelength of 400 nm of less than 0.003 AU. In some embodiments, the acetonitrile composition comprises an absorbance at a wavelength of 400 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition has a color (APHA) of less than 20 (e.g., less than 19, 18, 17, 16, 15, 14, 13, 12, 11, 10, 9, 8, 7, 6, 5, 4, 3, 2, or less than 1). In some embodiments, the acetonitrile composition has a color (APHA) of less than 15. In some embodiments, the acetonitrile composition has a color (APHA) of less than 10. In some embodiments, the acetonitrile composition has a color (APHA) of less than 5. In some embodiments, the acetonitrile composition has a color (APHA) of from about 1 to about 20, of from about 1 to about 15, of from about 1 to about 10, of from about 5 to about 15, or from about 5 to about 10.

The color of the acetonitrile composition may be measured according to the American Public Health Association (APHA) color scale.

In some embodiments, the acetonitrile composition provided herein is an oligonucleotide synthesis grade acetonitrile composition.

In some embodiments, oligonucleotide synthesis grade acetonitrile composition comprises purity of at least 99.8%. In some embodiments, oligonucleotide synthesis grade acetonitrile composition comprises a density at 20° C. of from about 0.780 g/mL to about 0.783 g/mL. In some embodiments, oligonucleotide synthesis grade acetonitrile composition comprises a refractive index at 20° C. of from about 1.343 to about 1.346. In some embodiments, oligonucleotide synthesis grade acetonitrile composition comprises less than 10 ppm of water. In some embodiments, oligonucleotide synthesis grade acetonitrile composition comprises an acidity (as CH₃COOH) of less than 0.001%. In some embodiments, oligonucleotide synthesis grade acetonitrile composition comprises a residue after evaporation of less than 0.001%. In some embodiments, oligonucleotide synthesis grade acetonitrile composition comprises an absorbance at a wavelength of 200 nm of less than 0.05 AU. In some embodiments, oligonucleotide synthesis grade acetonitrile composition comprises an absorbance at a wavelength of 210 nm of less than 0.03 nm. In some embodiments, oligonucleotide synthesis grade acetonitrile composition comprises an absorbance at a wavelength of 220 nm of less than 0.02 nm. In some embodiments, oligonucleotide synthesis grade acetonitrile composition comprises an absorbance at a wavelength of 230 nm of less than 0.01 nm. In some embodiments, oligonucleotide synthesis grade acetonitrile composition comprises an absorbance at a wavelength of 240 nm of less than 0.005 AU. In some embodiments, oligonucleotide synthesis grade acetonitrile composition comprises an absorbance at a wavelength of 260 nm of less than 0.005 AU. In some embodiments, oligonucleotide synthesis grade acetonitrile composition comprises an absorbance at a wavelength of 280 nm of less than 0.005 AU. In some embodiments, oligonucleotide synthesis grade acetonitrile composition comprises an absorbance at a wavelength of 400 nm of less than 0.005 AU. In some embodiments, oligonucleotide synthesis grade acetonitrile composition comprises a color (APHA) of less than 10.

In some embodiments, the acetonitrile composition provided herein is an ACS grade acetonitrile composition.

In some embodiments, ACS grade acetonitrile composition comprises a purity of at least 99.5%. In some embodiments, ACS grade acetonitrile composition comprises less than 3000 ppm (0.3%) water. In some embodiments, ACS grade acetonitrile composition comprises a residue after evaporation of less than 0.005%. In some embodiments, ACS grade acetonitrile composition comprises a color (APHA) of less than 10. In some embodiments, ACS grade acetonitrile composition comprises less than 8 μeq/g of titratable acid. In some embodiments, ACS grade acetonitrile composition comprises less than 0.6 μeq/g of titratable base. In some embodiments, the identity of ACS grade acetonitrile composition is confirmed by infrared spectroscopy.

In some embodiments, the acetonitrile composition provided herein is an HPLC grade acetonitrile composition.

In some embodiments, the HPLC grade acetonitrile composition comprises a purity of at least 99.9%. In some embodiments, the HPLC grade acetonitrile composition comprises an absorbance at a wavelength of 254 nm of less than 0.01 AU. In some embodiments, the HPLC grade acetonitrile composition comprises an absorbance at a wavelength of 220 nm of less than 0.02 AU. In some embodiments, the HPLC grade acetonitrile composition comprises an absorbance at a wavelength of 210 nm of less than 0.04 AU. In some embodiments, the HPLC grade acetonitrile composition comprises an absorbance at a wavelength of 205 nm of less than 0.05 AU. In some embodiments, the HPLC grade acetonitrile composition comprises an absorbance at a wavelength of 200 nm of less than 0.07 AU. In some embodiments, the HPLC grade acetonitrile composition comprises an absorbance at a wavelength of 295 nm of less than 0.15 AU. In some embodiments, the HPLC grade acetonitrile composition comprises an absorbance at a wavelength of 190 nm of less than 1 AU. In some embodiments, the HPLC grade acetonitrile composition comprises less than 8 μeq/g of titratable acid. In some embodiments, the HPLC grade acetonitrile composition comprises less than 0.6 μeq/g of titratable base. In some embodiments, the HPLC grade acetonitrile composition comprises less than 100 ppm (0.01%) water. In some embodiments, the HPLC grade acetonitrile composition comprises a residue after evaporation of less than 0.0001% (less than 1 ppm).

In some embodiments, the acetonitrile composition provided herein is an LC/MS grade acetonitrile. In some embodiments, the LC/MS grade acetonitrile composition has a purity of at least 99.8%.

In some embodiments, the acetonitrile composition is pharmaceutical grade acetonitrile. In some embodiments, pharmaceutical grade acetonitrile composition comprises a purity of at least 99.8%. In some embodiments, pharmaceutical grade acetonitrile composition comprises less than 2000 ppm water (0.2%).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1: Method of Preparing Bio-Acetonitrile

Acetonitrile may be prepared according to the scheme in FIG. 1.

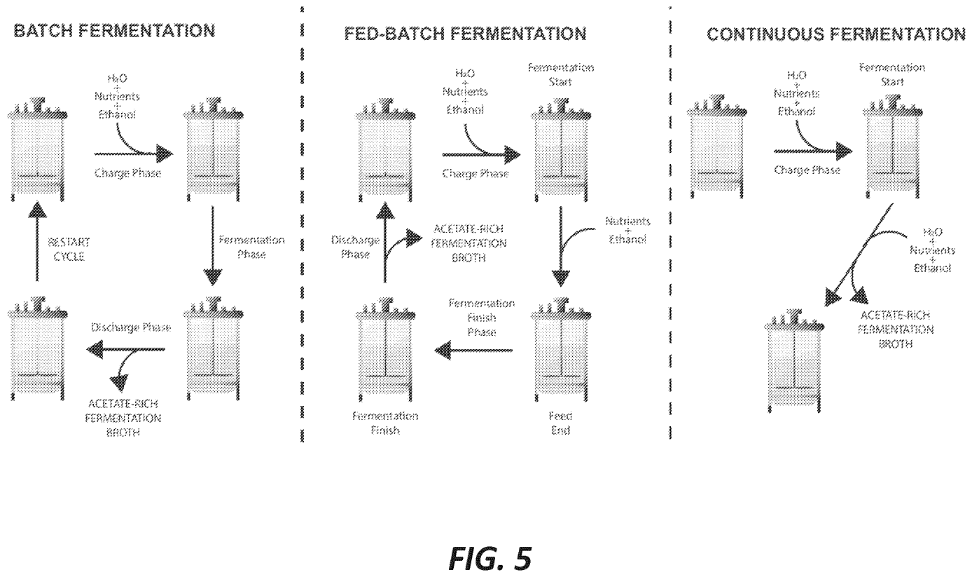

Ethanol was provided (106) and fermented to acetic acid according to vinegar or white vinegar production methods (102). The method includes semi-fed batch aerobic fermentation operated in a cycle including two modes, start-up or cycle. In start-up, a tank was filled with water, starter culture, and fed ethanol continuously until the acetic acid concentration reached 20 wt %. At this point, 60% of the fermenter was dropped to the well and the system was in cycle mode. The remaining 40% was retained and the fermenter was filled with water and the ethanol feeding process began again. The fermenters were continuously aerated at all times. Acetic acid was pumped from the fermenter to the acetic acid well. The acetic acid was pumped to the filter tank where it was recycled through the filter system until it was concentrated. Filtration removes rejected biomass. An example scheme for production of acetic acid from fermentation of ethanol is shown in FIG. 5.

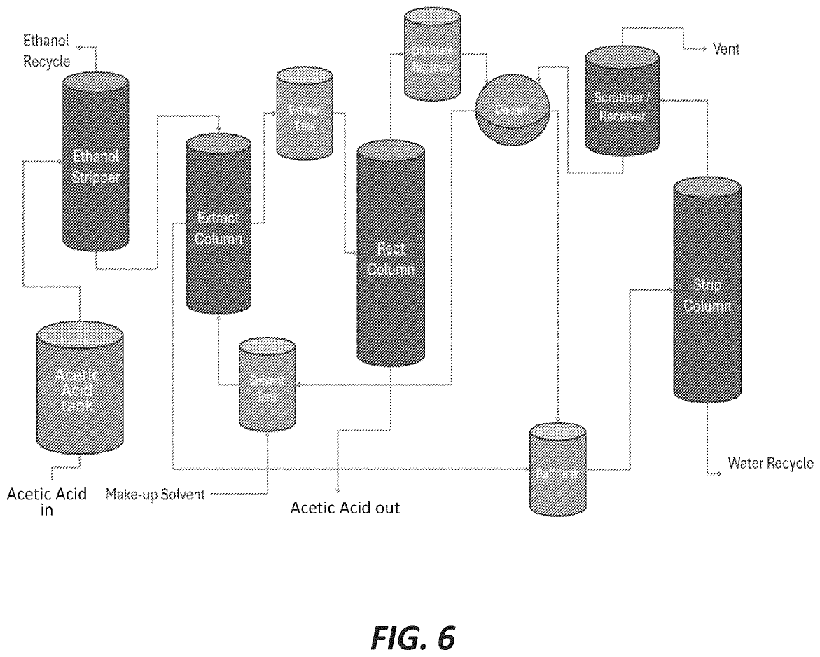

The filtered acetic acid was fed to an acetic acid purification section (103), FIG. 6. The acetic acid purification consists of a hybrid-extraction-distillation train. The first step was stripping of the acetic acid to remove ethanol (ethanol stripper of FIG. 6). This ethanol can be optionally recycled to the acetic acid fermentation process (107). The stripped acetic acid was then fed to an extraction column where it interacts with a methyl tert-butyl ether (MTBE) solvent stream (108) (extract column of FIG. 6). The MTBE-rich extract was fed to the rectification column from which a pure acetic acid stream was generated (rect. column of FIG. 6). The water-rich raffinate was fed to the water stripping column, where residual MTBE was vaporized and removed from the water. The water, which leaves the water stripping column (strip column of FIG. 6), may be recycled back to the fermentation section (107). The distillate of both the rectification column and the water stripping column were combined, cooled, and sent to a decanter where the organic and aqueous phases were separated and sent to the extraction column and the water stripping column, respectively. The water recovered from the water stripping column optionally either underwent wastewater treatment (110) or was recycled for use during the acetic acid fermentation.

The resulting (e.g., glacial) acetic acid was preheated, mixed, and fed to the reactor (104) with ammonia (109) and optionally the recycled ammonia stream (112). The reactor was a fixed bed catalyst operated at a temperature between 280° C.-400° C. (e.g., 290° C.-320° C.) and a pressure of 1-10 bar (e.g., 1-4 bar). The reaction was endothermic and the reactor must be heated to maintain favorable reaction temperatures. In some instances, side reactions become more pronounced at higher temperatures, and reactor fouling occurs at lower temperatures due to incomplete reaction. The reaction of the acetic acid and ammonia is shown in FIG. 3. The resulting crude reactor product contains ammonia, water, acetonitrile, acetone, carbon dioxide, and minor impurities.

The crude reactor product was purified (105). Ammonia was distilled off from the crude reactor product via a water scrubbing distillation column and the nominally pure ammonia stream was optionally recycled to the reactor feed (112). The crude reactor product was mixed with an oxidant, such as potassium permanganate, under acidic, neutral, or basic conditions. Oxidation of acetone and other impurities may allow for further purification of the acetonitrile. Excess oxidant was quenched (e.g., with sodium thiosulfate), the solution was returned to neutral pH (e.g., by adding sulfuric acid if the oxidation reaction was conducted under basic conditions), and solids formed during the reaction were filtered. The oxidized, quenched, neutralized, and filtered crude product was fed to a modified pressure swing distillation for water (and impurities) and acetonitrile separation.

During the acetonitrile purification (105), the crude reactor product was cooled and fed to an ammonia recovery column, the ammonia and carbon dioxide are the distillate products. The ammonia and carbon dioxide vapor were scrubbed in a multi-stage water scrubbing column before the nominally pure ammonia was recycled to the reactor feed. The aqueous carbon dioxide stream was partially bled off as waste and recycled to the scrubber. The water, acetonitrile, and acetone was fed to a continuously stirred tank reactor, where an oxidizing agent or catalysts such as potassium permanganate was fed to the system. This reactor has an adequately long residence time (as little as 20 minutes or up to 2 hours or more) for acetone and other impurities (e.g., unsaturated nitriles) to fully react. The reactor effluent was fed to a distillation column which separates the oxidation reaction products from the crude reactor product. The purified reactor product was fed to the pressure swing distillation apparatus. First, this stream enters the low pressure column, which operates at atmospheric pressure or below atmospheric pressure. There is a secondary feed stream to this column which is the azeotrope recycle from the high pressure column. The bottoms product of the low pressure column is nominally pure water. The distillate of the low pressure column is near the water and acetonitrile azeotrope, although this can be distorted due to the presence of impurities (primarily acetone and ammonia). The low pressure column distillate was chilled, compressed to higher pressure, and fed to the high pressure column. The bottoms of the high pressure column are nominally pure acetonitrile. The distillate of the high pressure column is the higher pressure water and acetonitrile azeotrope, with additional impurities (primarily acetone and ammonia). The high pressure distillate was fed to a water stripping column which purges acetone and any residual ammonia and/or carbon dioxide (or other volatiles) in the distillate (111). The bottoms of the water stripping column was nominally water and acetonitrile at the high pressure azeotrope, which was recycled to the low pressure column.

The nominally pure acetonitrile was then polished to meet requisite purity specifications (105). Purity specifications may include technical grade, laboratory grade, USP grade, ACS grade, pharma grade, or oligonucleotide synthesis grade. This was accomplished via two sequential polishing steps. The acetonitrile was passed through an activated carbon bed which removes the aromatics (below ~1 ppm). This stream was passed through a bed of 3 A molecular sieves to remove water. Finally, any remaining water and trace polar impurities were removed via an activated alumina column to below 10 ppm.

The polished acetonitrile (101) was (e.g., immediately) packaged and stored.

### Example 2: Method of Preparing Bio-Acetonitrile

Acetonitrile may be prepared according to the scheme in FIG. 2.

Acetic acid may be provided (202) as produced by any suitable method. The acetic acid may be purified or require purification in subsequent steps.

The acetic acid was fed to an acetic acid purification section (203), FIG. 6. The acetic acid purification consists of a hybrid-extraction-distillation train. The first step was stripping of the acetic acid to remove ethanol (ethanol stripper of FIG. 6). The stripped acetic acid was then fed to an extraction column where it interacted with a methyl tert-butyl ether (MTBE) solvent stream (208) (extract column of FIG. 6). The MTBE-rich extract was fed to the rectification column from which a pure acetic acid stream is generated (rect. column of FIG. 6). The water-rich raffinate was fed to the water stripping column, where residual MTBE was vaporized and removed from the water. The distillate of both the rectification column and the water stripping column were combined, cooled, and sent to a decanter where the organic and aqueous phases were separated and sent to the extraction column and the water stripping column, respectively. The water recovered from the water stripping column optionally either underwent wastewater treatment or was recycled for use during the acetic acid fermentation.

The resulting (e.g., glacial) acetic acid was preheated, mixed, and fed to the reactor (204) with ammonia (208) and optionally the recycled ammonia stream (211). The reactor was a fixed bed catalyst operated at a temperature between 280° C.-400° C. (e.g., 290° C.-320° C.) and a pressure of 1-10 bar (e.g., 1-4 bar). The reaction was endothermic and the reactor must be heated to maintain favorable reaction temperatures. In some instances, side reactions become more pronounced at higher temperatures, and reactor fouling occurs at lower temperatures due to incomplete reaction. The reaction of the acetic acid and ammonia is shown in FIG. 3. The resulting crude reactor product contains ammonia, water, acetonitrile, acetone, carbon dioxide, and minor impurities.

The crude reactor product was purified (205). Ammonia was distilled off from the crude reactor product via a water scrubbing distillation column and the nominally pure ammonia stream was optionally recycled to the reactor feed (211). The crude reactor product was mixed with an oxidant, such as potassium permanganate, under acidic, neutral, or basic conditions. Oxidation of acetone and other impurities may allow for further purification of the acetonitrile. Excess oxidant was quenched (e.g., with sodium thiosulfate), the solution was returned to neutral pH (e.g., by adding sulfuric acid if the oxidation reaction was conducted under basic conditions), and solids formed during the reaction were filtered. The oxidized, quenched, neutralized, and filtered crude product was fed to a modified pressure swing distillation for water (and impurities) and acetonitrile separation.

During the acetonitrile purification (205), the crude reactor product was cooled and fed to an ammonia recovery column, the ammonia and carbon dioxide are the distillate products. The ammonia and carbon dioxide vapor was scrubbed in a multi-stage water scrubbing column before the nominally pure ammonia is recycled to the reactor feed. The aqueous carbon dioxide stream was partially bled off as waste and recycled to the scrubber. The water, acetonitrile, and acetone are fed to a continuously stirred tank reactor, where an oxidizing agent or catalysts such as potassium permanganate is fed to the system. This reactor has an adequately long residence time (as little as 20 minutes or up to 2 hours or more) for acetone and other impurities (e.g., unsaturated nitriles) to fully react. The reactor effluent was fed to a distillation column which separates the oxidation products from the crude reactor product. The distillate of this column was fed to the pressure swing distillation apparatus. First, this stream enters the low pressure column, which operates at atmospheric pressure or below atmospheric pressure. There is a secondary feed stream to this column which is the azeotrope recycle from the high pressure column. The bottoms product of the low pressure column is nominally pure water. The distillate of the low pressure column was near the water and acetonitrile azeotrope, although the exact composition can be distorted due to the presence of impurities (primarily acetone). The low pressure column distillate was chilled, compressed to higher pressure, and fed to the high pressure column. The bottoms of the high pressure column are nominally pure acetonitrile. The distillate of the high pressure column was the higher pressure water and acetonitrile azeotrope, with additional impurities (primarily acetone). The high pressure distillate was fed to a water stripping column which purges acetone and any residual ammonia and/or carbon dioxide (or other volatiles) in the distillate (210). The bottoms of the water stripping column was nominally water and acetonitrile at the high pressure azeotrope, which was recycled to the low pressure column.

The nominally pure acetonitrile was then polished to meet requisite purity specifications (105). Purity specifications may include technical grade, laboratory grade, USP grade, ACS grade, pharma grade, or oligonucleotide synthesis grade. This was accomplished via two sequential polishing steps. The acetonitrile was passed through an activated carbon bed which removes the aromatics (below ~1 ppm) and a molecular sieve bed to remove the majority of the water. Finally, any remaining water and other polar impurities were removed via an activated alumina column to below 10 ppm.

The polished acetonitrile (201) was (e.g., immediately) packaged and stored.

1. A method of making an acetonitrile composition, the method comprising:
(a) providing a biologically produced acetonitrile precursor;
(b) purifying the biologically produced acetonitrile precursor;
(c) reacting the biologically produced acetonitrile precursor with a nitrogen source and a catalyst to provide crude acetonitrile; and
(d) purifying the crude acetonitrile to provide the acetonitrile composition, wherein the reacting produces no more than 20 wt % of organic products other than acetonitrile, and wherein the acetonitrile composition comprises less than 2 wt % of impurities.

2. A method of making an acetonitrile composition, the method comprising:
(a) providing a biologically produced acetonitrile precursor;
(b) purifying the biologically produced acetonitrile precursor;
(c) reacting the biologically produced acetonitrile precursor with a nitrogen source and a catalyst to provide crude acetonitrile; and
(d) purifying the crude acetonitrile to provide the acetonitrile composition, wherein the method does not generate hydrogen cyanide.

3-8. (canceled)

9. The method of claim 1, wherein the acetonitrile composition comprises an absorbance of less than 0.1 absorbance units (AU) at a wavelength of 200 nm, 210 nm, 220 nm, 230 nm, 240 nm, 260 nm, and 280 nm.

10-22. (canceled)

23. The method of claim 1, wherein the acetonitrile composition comprises an absorbance of less than 0.01 AU at a wavelength of 400 nm.

24. (canceled)

25. The method of claim 1, wherein the acetonitrile composition comprises no more than 20 ppm water.

26. (canceled)

27. The method of claim 1, wherein the acetonitrile composition is anhydrous acetonitrile.

28. The method of claim 1, wherein the biologically produced acetonitrile precursor is produced by fermentation.

29. (canceled)

30. (canceled)

31. The method of claim 1, wherein the purifying the biologically produced acetonitrile precursor comprises liquid-liquid extraction, distillation, or a combination thereof.

32. The method of claim 31, wherein the liquid-liquid extraction comprises contacting the biologically produced acetonitrile precursor with an organic solvent.

33. The method of claim 32, wherein the organic solvent is ethyl acetate, butyl acetate, diethyl ether, dichloromethane, toluene, chloroform, methyl tert-butyl ether, toluene, chloroform, hexane, benzene, acetone, or a combination thereof.

34. The method of claim 1, wherein the biologically produced acetonitrile precursor is acetic acid.

35. (canceled)

36. (canceled)

37. The method of claim 1, wherein the catalyst is aluminum oxide, titanium dioxide, zirconium dioxide, or tungsten oxide or a combination thereof.

38. (canceled)

39. (canceled)

40. (canceled)

41. The method of claim 1, wherein the reacting comprises heating to a temperature of from about 250° C. to about 350° C.

42. The method of claim 1, wherein the nitrogen source is ammonia.

43. The method of claim 1, wherein the reacting comprises a pressure of from about 0.8 atm to about 5 atm.

44. (canceled)

45. The method of claim 1, wherein the purifying the crude acetonitrile comprises distillation, oxidation, polishing, or a combination thereof.

46. (canceled)

47. The method of claim 45, wherein the excess nitrogen source is recycled in (c).

48. The method of claim 1, wherein the purifying comprises use of an oxidant.

49. (canceled)

50. The method of claim 48, wherein the oxidant is potassium permanganate, ozone, or potassium superoxide.

51. (canceled)

52. (canceled)

53. The method of claim 1, wherein the purifying the crude acetonitrile is completed in basic conditions.

54. The method of claim 45, wherein the polishing of the crude acetonitrile comprises contacting the crude acetonitrile with a zeolite, ion-exchange resin, activated alumina, activated carbon, or a combination thereof.

55. The method of claim 45, wherein the polishing of the crude acetonitrile comprises contacting the crude acetonitrile with silica, calcium sulfate, molecular sieves, a zeolite, alumina, or magnesium sulfate.

56. The method of claim 1, wherein the method produces the acetonitrile composition in a quantity of at least 100 L.

57-59. (canceled)

60. The method of claim 1, wherein the method does not produce acrylonitrile.

61. (canceled)

62. The method of claim 1, wherein the acetonitrile composition comprises a reduction in carbon intensity (CI) as compared to an acetonitrile composition produced using the SOHIO process.

63. (canceled)

64. A composition comprising acetonitrile, produced by the method of claim 1.

65. The composition of claim 64, wherein the composition comprises: at least 98 wt % acetonitrile derived from a biologically produced precursor and less than 10 ppm water and does not comprise acetonitrile derived from a fossil fuel source.

66-68. (canceled)

69. The composition of claim 64, wherein the composition comprises an absorbance of less than 0.1 absorbance units (AU) at a wavelength of 200 nm, 210 nm, 220 nm, 230 nm, 240 nm, 260 nm, and 280 nm.

70-82. (canceled)

83. The composition of claim 64, wherein the composition comprises an absorbance of less than 0.01 AU at a wavelength of 400 nm.

84. (canceled)

85. The composition of claim 64, wherein the composition comprises anhydrous acetonitrile.
